(19) United States
(12) Patent Application Publication
(10) Pub. No.: US 2025/0006301 A1
(43) Pub. Date: Jan. 2, 2025

(54) METHODS AND SYSTEMS FOR GENOTYPING BY SANGER-BASED DNA SEQUENCING

(71) Applicant: LIFE TECHNOLOGIES CORPORATION, Carlsbad, CA (US)

(72) Inventor: Edgar Hans SCHREIBER, Redwood City, CA (US)

(21) Appl. No.: 18/700,683

(22) PCT Filed: Oct. 17, 2022
(86) PCT No.: PCT/US22/78242

§ 371 Date: Apr. 11, 2024

Related U.S. Application Data
(60) Provisional application No. 63/256,487, filed on Oct. 15, 2021.

(51) Int. Cl. G16B 20/20; G16B 30/10

(52) U.S. Cl. CPC G16B 20/20; G16B 30/10

(57) ABSTRACT

Methods and systems are provided for genotyping a gene sequence. The method comprises obtaining first genotyping call data representing a query gene sequence. A numerical score is assigned to each of a plurality of allele calls of second genotyping call data by matching the first genotyping call data with the second genotyping call data. the second genotyping call data representing a plurality of candidate gene sequences. A match score is determined for each of the plurality of candidate gene sequences based on the numerical score assigned to each of the plurality of allele calls of the second genotyping call data, and a genotyping call is made for the query gene sequence based on a highest match score from among the match score determined for each of the plurality of candidate gene sequences.

### CROSS-REFERENCE TO RELATED APPLICATIONS

This application claims the benefit of U.S. provisional application No. 63/256,487 filed on Oct. 15, 2021. The entire contents of that application are hereby incorporated by reference, to the extent allowed in applicable jurisdictions.

### TECHNICAL FIELD

This disclosure relates generally to DNA sequencing at specific positions within the genome of an individual, and more specifically to inventive methods for genotyping gene sequences and systems configured for genotyping gene sequences.

### BACKGROUND

DNA sequencing is the process of determining a nucleic acid sequence—the order of nucleotides in DNA. It includes any method or technology that is used to determine the order of the four base nucleotides: adenine, guanine, cytosine, and thymine.

DNA sequencing is at the core of modern molecular biology, and the advent of rapid DNA sequencing methods has accelerated medical research and discovery in applied fields such as medical diagnosis, therapeutics, biotechnology, and virology. DNA sequencing can be used for a variety of applications, including de novo sequencing of genomes (i.e., the generation of the sequence of a DNA molecule without any prior information about the sequence.); detection of variants (SNPs) and mutations; biological identification; confirmation of clone constructs; detection of methylation events; gene expression studies; and detection of copy number variation. DNA sequencing can also be used for ABO blood group matching, e.g., between a blood donor and a recipient.

Sanger-based DNA sequencing (also referred to herein as Sanger dideoxy sequencing or Sanger sequencing) is a method of DNA sequencing that is based on the selective incorporation of chain-terminating dideoxynucleotides by DNA polymerase (the enzyme responsible for forming new copies of DNA) during in vitro DNA replication. Sanger sequencing has been in use since the 1970's, and it remains in wide use for smaller-scale tasks like the sequencing of single genes, cloned plasmids, expression constructs or PCR products. For example, Sanger sequencing is often used to study a small subset of genes linked to a defined phenotype, such as an individual's blood type.

The Sanger sequencing process takes advantage of the ability of DNA polymerase to incorporate 2′,3′-dideoxynucleotides—nucleotide base analogs that lack the 3′-hydroxyl group essential in phosphodiester bond formation. Four separate reactions are set up, each containing radioactively labeled nucleotides and either of the four dideoxynucleotides: ddA, ddC, ddG, or ddT. DNA polymerase adds a deoxynucleotide or the corresponding 2′,3′-dideoxynucleotide at each step of chain extension. Whether a deoxynucleotide or a dideoxynucleotide is added depends on the relative concentration of both molecules. If a deoxynucleotide (A, C, G, or T) is added to the 3′ end, chain extension can continue. However, if a dideoxynucleotide (ddA, ddC, ddG, or ddT) is added to the 3′ end, the chain extension terminates. Sanger dideoxy sequencing results in the formation of extension products of various lengths terminated with dideoxynucleotides at the 3′ end.

Capillary electrophoresis is used to separate the extension products resulting from Sanger dideoxy sequencing. During capillary electrophoresis, the molecules are injected by an electrical current into a glass capillary filled with a gel polymer, and an electrical field is applied so that the negatively charged DNA fragments move toward the positive electrode. The speed at which a DNA fragment moves through the capillary medium is inversely proportional to its molecular weight. In practice, the process of capillary electrophoresis can separate the extension products by size at a resolution of one base.

Like Sanger sequencing, fluorescence-based cycle sequencing requires a DNA template, a sequencing primer, a thermal stable DNA polymerase, deoxynucleoside triphosphates/deoxynucleotides (dNTPs), dideoxynucleoside triphosphates/dideoxynucleotides (ddNTPs), and a buffer. But unlike Sanger sequencing, which uses radioactive material, cycle sequencing uses fluorescent dyes to label the extension products. The components are combined in a reaction that is subjected to cycles of annealing, extension, and denaturation in a thermal cycler. Thermal cycling the sequencing reactions creates and amplifies extension products that are terminated by one of the four dideoxynucleotides. The ratio of deoxynucleotides to dideoxynucleotides is optimized to produce a balanced population of long and short extension products.

Fluorescence-based cycle sequencing can be an extension and refinement of Sanger sequencing. In general, a combined Sanger and fluorescence-based cycle sequence workflow can include DNA template preparation (e.g., by PCR); cycle sequencing; purification of extension products after cycle sequencing; capillary electrophoresis; and data analysis (e.g., applying analysis profiles, running analyses, and allowing a review of data).

While modern DNA sequencing workflows can facilitate accurate genotyping, certain processes for the determination of common and rare genotypes with possible weak phenotypes, which may evade correct typing by serology, continue to present technical challenges that can adversely affect the goal of obtaining useful and clinically important test results. In particular, the mixed sequencing traces from heterozygous alleles can often be challenging to decipher for genotyping complex loci like the ABO (blood group) gene, which can be critical for same ABO blood group matching between donor and recipient (e.g., to prevent adverse reaction or graft dysfunction due to an ABO genotype mismatch in organ transplantation), providing for reasonable organ allocation, and informed selection of optimal transfusion therapies, including for the cis-AB blood group.

### SUMMARY

Various computer-implemented systems, methods, and articles of manufacture for genotyping a gene sequence, are described herein that can improve the accuracy of genotyping results with respect to the various challenges mentioned above.

In one embodiment, a method of genotyping a gene sequence is provided. The method comprises obtaining first genotyping call data representing a query gene sequence. A numerical score is assigned to each of a plurality of allele calls of second genotyping call data by matching the first genotyping call data with the second genotyping call data, the second genotyping call data representing a plurality of candidate gene sequences. A match score is determined for each of the plurality of candidate gene sequences based on the numerical score assigned to each of the plurality of allele calls of the second genotyping call data, and a genotyping call is made for the query gene sequence based on a highest match score from among the match scores determined for each of the plurality of candidate gene sequences. The determining of the match score for each of the plurality of candidate gene sequences may comprise summing numerical scores assigned to each of the plurality of allele calls of the second genotyping call data.

In some embodiments, obtaining the first genotyping call data may comprise obtaining Sanger-based DNA sequencing data representing the query gene sequence, aligning the Sanger-based DNA sequencing data representing the query gene sequence with a reference gene sequence, making an additional genotyping call for each of a plurality of alleles of the query gene sequence based on the aligned Sanger-based DNA sequencing data, and translating the additional genotyping call for each of the plurality of alleles into a code representing the query gene sequence. The code may comprise an International Union of Pure and Applied Chemistry (IUPAC) code, and a heterozygous or homozygous deletion code.

In some embodiments, the making of the additional genotyping call may comprise generating an electropherogram report of base calls for each of the plurality of alleles of the query gene sequence based on the aligned Sanger-based DNA sequencing data using at least one base caller algorithm, and verifying the base calls for each of the plurality of alleles of the query gene sequence based on an analysis of the electropherogram report.

In some embodiments, the method may comprise assigning a first numerical value of “1” if there is a positive match between an allele call of the first genotyping call data and a corresponding allele call of the second genotyping call data, and a second numerical value of “0” if there is a non-positive match between the allele call of the first genotyping call data and the corresponding allele call of the second genotyping call data.

In some embodiments, the method may comprise generating a look-up table comprising the second genotyping call data, where the look-up table comprises a list of codes representing each of the plurality of candidate gene sequences, and where the list of codes comprises International Union of Pure and Applied Chemistry (IUPAC) codes, and a heterozygous or homozygous deletion code.

In some embodiments, the matching of the first genotyping call data with the second genotyping call data may comprise aligning each allele position of the first genotyping call data with corresponding allele positions of the second genotyping call data, and comparing, at each of the corresponding allele positions, allele calls of the first genotyping call data with the plurality of allele calls of the second genotyping call data.

Various objects, features, aspects, and advantages of the inventive subject matter will become more apparent from the following specification, along with the accompanying drawings in which like numerals represent like components.

### BRIEF DESCRIPTION OF THE DRAWINGS

The patent or application file contains at least one drawing executed in

color. Copies of this patent or patent application publication with color drawing(s) will be provided by the Office upon request and payment of the necessary fee.

FIG. 1 illustrates a block diagram of a system configured for genotyping according to an embodiment, the system including a PCR apparatus, a capillary electrophoresis apparatus, and a genotyping data analyzer.

FIG. 2 illustrates a block diagram of a genotyping data analyzer according to an embodiment.

FIG. 3 illustrates an exemplary functional block diagram of a genotyping workflow process for genotyping a query gene sequence according to an embodiment.

FIG. 4 illustrates a diagram and genotyping call data representing alleles of interest in a query gene sequence.

FIG. 5 illustrates a diagram of genomic loci and primers used for amplifying alleles of interest in a query gene sequence.

FIG. 6 illustrates a diagram of PCR primers applied to amplify alleles of interest in a query gene sequence.

FIG. 7 illustrates an example DNA sequence that may be applied as an amplicon insert in an ABO genotyping analysis.

FIG. 8 illustrates an interface display of data settings for a reference gene sequence.

FIG. 9 illustrates a dedicated tab for visualization of variants for alleles of interest in a query gene sequence.

FIG. 10 illustrates an interface display of an assembly overview of query gene sequence traces to a reference gene sequence.

FIG. 11 illustrates an interface display corresponding to alleles of interest obtained after a KBTM Basecaller generated base call.

FIG. 12 illustrates an interface display of allele call data corresponding to alleles of interest in a query gene sequence.

FIG. 13 illustrates an interface display of allele call data corresponding to alleles of interest in a query gene sequence.

FIG. 14A illustrates a genotyping call data look-up table representing a plurality of candidate gene sequences.

FIG. 14B illustrates genotyping call data representing a candidate gene sequence.

FIG. 15 illustrates an interface display of numerical scores assigned to each of a plurality of allele calls of second genotyping call data representing a plurality of candidate gene sequences and a match score determined for each of the plurality of candidate gene sequences.

FIG. 16 is a flowchart illustrating a method genotyping a gene sequence according to various embodiments.

FIG. 17 is a flowchart illustrating a method for obtaining the first genotyping call data.

FIG. 18 is a flowchart illustrating a method for making the additional genotyping call in accordance with various embodiments.

FIG. 19 is a flowchart illustrating a method for matching of the first genotyping call data with the second genotyping call data in accordance with various embodiments.

FIG. 20 illustrates a block diagram of a computer system that can be used for implementing one or more aspects of the various embodiments.

While the invention is described with reference to the above drawings, the drawings are intended to be illustrative, and other embodiments are consistent with the spirit, and within the scope, of the invention.

### DETAILED DESCRIPTION

To provide a more thorough understanding of the present invention, the following description sets forth numerous specific details, such as specific configurations, parameters, examples, and the like. It should be recognized, however, that such description is not intended as a limitation on the scope of the present invention but is intended to provide a better description of the exemplary embodiments.

Throughout the specification and claims, the following terms take the meanings explicitly associated herein, unless the context clearly dictates otherwise:

The phrase “in one embodiment” as used herein does not necessarily refer to the same embodiment, though it may. Thus, as described below, various embodiments of the invention may be readily combined, without departing from the scope or spirit of the invention.

As used herein, the term “or” is an inclusive “or” operator and is equivalent to the term “and/or,” unless the context clearly dictates otherwise.

The term “based on” is not exclusive and allows for being based on additional factors not described, unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networked environment where two or more components or devices are able to exchange data, the terms “coupled to” and “coupled with” are also used to mean “communicatively coupled with”, possibly via one or more intermediary devices.

In addition, throughout the specification, the meaning of “a”, “an”, and “the” includes plural references, and the meaning of “in” includes “in” and “on”.

Although some of the various embodiments presented herein constitute a single combination of inventive elements, it should be appreciated that the inventive subject matter is considered to include all possible combinations of the disclosed elements. As such, if one embodiment comprises elements A, B, and C, and another embodiment comprises elements B and D, then the inventive subject matter is also considered to include other remaining combinations of A, B, C, or D, even if not explicitly discussed herein. Further, the transitional term “comprising” means to have as parts or members, or to be those parts or members. As used herein, the transitional term “comprising” is inclusive or open-ended and does not exclude additional, unrecited elements or method steps.

Throughout the following disclosure, numerous references may be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable medium storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, a circuit-switched network, the Internet, LAN, WAN, VPN, or other type of network.

As used in the description herein and throughout the claims that follow, when a system, engine, server, device, module, or other computing element is described as being configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

It should be noted that any language directed to a computer should be read to include any suitable combination of computing devices or network platforms, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

In various embodiments, the devices, instruments, systems, and methods described herein may be used to detect one or more types of biological components of interest. These biological components of interest may be any suitable biological target including, but are not limited to, DNA sequences (including cell-free DNA), RNA sequences, genes, oligonucleotides, molecules, proteins, biomarkers, cells (e.g., circulating tumor cells), or any other suitable target biomolecule.

In various embodiments, such biological components may be used in conjunction with various PCR and capillary electrophoresis methods and systems in applications such as genotyping and rare allele detection.

According to various embodiments, the present disclosure may be directed to devices, instruments, systems, and methods for measuring or quantifying a biological reaction of interest, and therefore a corresponding biological component of interest, for a large number of small volume samples.

While generally applicable to digital quantification such as PCR, it should be recognized that any other suitable quantification method may be used in accordance with various embodiments described herein. Suitable PCR methods include, but are not limited to, digital PCR, real-time PCR, allele-specific PCR, asymmetric PCR, ligation-mediated PCR, multiplex PCR, nested PCR, quantitative PCR, genome walking, and bridge PCR, for example.

As used herein, thermal cycling may include using a thermal cycler, isothermal amplification, thermal convention, infrared mediated thermal cycling, or helicase dependent amplification, for example.

According to various embodiments, detection of a target may be, but is not limited to, fluorescence detection, detection of positive or negative ions, pH detection, voltage detection, or current detection, alone or in combination, for example.

Various embodiments described herein may be suited for digital PCR (dPCR). In digital PCR, a solution containing a relatively small number of a target analyte, e.g., a polynucleotide or nucleotide sequence, may be subdivided into a large number of small test samples, such that each sample generally contains either one molecule of the target analyte, e.g., a nucleotide sequence, or none of the target. When the samples are subsequently thermally cycled in a PCR protocol, procedure, or experiment, the sample containing the target are amplified and produce a positive detection signal, while the samples containing no target are not amplified and produce no detection signal. Using Poisson statistics, the number of targets in the original solution may be correlated to the number of samples producing a positive detection signal.

Various embodiments of the present disclosure are directed to Sanger-based DNA sequencing. In general, Sanger-based DNA sequencing (also referred to herein as Sanger dideoxy sequencing or Sanger sequencing) requires a DNA template, a sequencing primer, DNA polymerase, deoxynucleotides (dNTPs), dideoxynucleotides (ddNTPs), and reaction buffer. Four separate reactions are set up, each containing radioactively labeled nucleotides and either of the four dideoxynucleotides (ddA, ddC, ddG, or ddT). Annealing, labeling, and termination steps are performed on separate heat blocks. DNA synthesis is performed at ˜37° C., the temperature at which DNA polymerase has the optimal enzyme activity. DNA polymerase adds a deoxynucleotide or the corresponding 2′,3′-dideoxynucleotide at each step of chain extension. Whether a deoxynucleotide or a dideoxynucleotide is added depends on the relative concentration of both molecules. If a deoxynucleotide (A, C, G, or T) is added to the 3′ end, chain extension can continue. However, if a dideoxynucleotide (ddA, ddC, ddG, or ddT) is added to the 3′ end, chain extension terminates. Sanger dideoxy sequencing results in the formation of extension products of various lengths terminated with dideoxynucleotides at the 3′ end.

While various embodiments of the present disclosure are directed to Sanger dideoxy sequencing, embodiments of the present invention are not limited thereto. Other forms of DNA sequencing such as large-scale sequencing, and other high-throughput sequencing (e.g., next-generation sequencing, and sequencing by ligation (also referred to as “SOLID SEQUENCING®”), polony sequencing, and shotgun sequencing may be used.

According to various embodiments, capillary electrophoresis may be used to separate the extension products resulting from Sanger dideoxy sequencing. During capillary electrophoresis, the molecules are injected by an electrical current into a glass capillary filled with a gel polymer, and an electrical field is applied so that the negatively charged DNA fragments move toward the positive electrode. The speed at which a DNA fragment moves through the capillary medium is inversely proportional to its molecular weight. In practice, the process of capillary electrophoresis can separate the extension products by size at a resolution of one base.

Various embodiments described herein may be suited for fluorescence-based cycle sequencing used as an extension and refinement of Sanger dideoxy sequencing. In general, a combined Sanger and fluorescence-based cycle sequence workflow can include DNA template preparation (e.g., by PCR); cycle sequencing; purification of extension products after cycle sequencing; capillary electrophoresis; and data analysis (e.g., applying analysis profiles, running analyses, and allowing a review of data).

One should appreciate that the disclosed techniques provide many advantageous technical effects including automated methods for genotyping a gene sequence using a system including an analyte detection (e.g., a PCR) apparatus, a capillary electrophoresis apparatus, and a genotyping data analyzer. The techniques described herein employ logic to automate various processes. Further, the disclosed techniques have been designed to support data accuracy and allow for processing data algorithms and complex permutations on a scale and speed that cannot be achieved using manual human effort.

It should also be appreciated that the following specification is not intended as an extensive overview, and as such, concepts may be simplified in the interests of clarity and brevity.

A method developed for genotyping a gene sequence using PCR, fluorescence-based cycle sequencing, Sanger sequencing, and capillary electrophoresis techniques is described herein. In various embodiments, the method entails bi-directional Sanger sequencing of PCR-generated amplicons and analyzing the resulting sequence trace files. In various embodiments, the method further entails aligning and matching a query gene sequence with a plurality of candidate gene sequences using one or more find operations in a look-up table including a list of codes representing the plurality of candidate gene sequences.

The method provides advantages over previous genotyping methods, which in some cases have required manual human effort, by providing various improved techniques, including techniques for deciphering the mixed sequencing traces from heterozygous alleles that can often be challenging for genotyping complex loci like the ABO (blood group) gene. These improved techniques can be employed in various medical research and clinical applications including, for example, same ABO blood group matching between donor and recipient (e.g., to prevent an adverse reaction or graft dysfunction due to an ABO genotype mismatch in organ transplantation), providing for reasonable organ allocation, and informed selection of optimal transfusion therapies, particularly for the cis-AB blood group.

It should be noted, however, that while the example of genotyping call data corresponding to ABO alleles is used throughout this description, the various embodiments described herein are not limited to determining major ABO blood types. Rather, the various embodiments described herein can apply generally to making a genotyping call for a query gene sequence based on a highest match score from among the match scores determined for each of a plurality of candidate gene sequences.

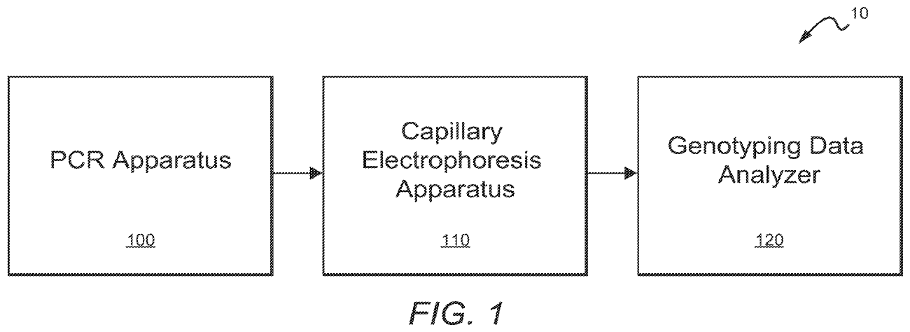

FIG. 1 illustrates a block diagram of a system 10 configured for genotyping according to an embodiment. System 10 includes a PCR apparatus 100, a capillary electrophoresis apparatus 110, and a genotyping data analyzer 120.

In some embodiments, PCR apparatus 100 is an apparatus configured to perform at least one of real-time PCR, allele-specific PCR, asymmetric PCR, ligation-mediated PCR, multiplex PCR, nested PCR, quantitative PCR, genome walking, and bridge PCR.

In some embodiments, PCR apparatus 100 is an apparatus configured to perform digital PCR or a digital PCR apparatus. As described above, digital PCR (dPCR) uses a solution including a relatively small number of a target analyte, e.g., a polynucleotide or nucleotide sequence template DNA (or RNA), fluorescence-quencher probes, primers, and a PCR master mix comprising DNA polymerase and reaction buffers at optimal concentrations. The solution is partitioned into a large number of small test samples, e.g., tens of thousands of microchambers disposed within a microfluidic array plate. Thermal cycling is subsequently performed with respect to array of partitions using PCR apparatus 100 to produce a PCR amplification, e.g., of a query gene sequence, in preparation for Sanger sequencing.

A Sanger sequencing workflow may include cycle sequencing, and purification (e.g., using a purification kit as sold under the name BigDye® Xterminator™ Purification Kit) of extension products after cycle sequencing. Purification results in the removal of unincorporated terminators and/or salts from the cycle sequencing reactions.

In an embodiment, PCR apparatus 100 is used for both PCR amplification and Sanger sequencing of the query gene sequence.

In another embodiment, PCR apparatus 100 is used for PCR amplification, and system 10 further includes a DNA sequencer (not shown), which is an instrument (e.g., the analyzer sold under the name Applied Biosystems™ SeqStudio® Genetic Analyzer) configured for performing sequencing reactions on the PCR amplification of the query gene sequence.

After the sequencing reactions are purified, the array plate is transferred to capillary electrophoresis apparatus 110 (e.g., the apparatus sold under the name Applied Biosystems™ 3500xL Genetic Analyzer) for capillary electrophoresis. The resulting sequencing files (e.g.,.ab1 digital files) containing the nucleotide sequences of the processed samples may be analyzed using genotyping data analyzer 120 to make a genotyping call.

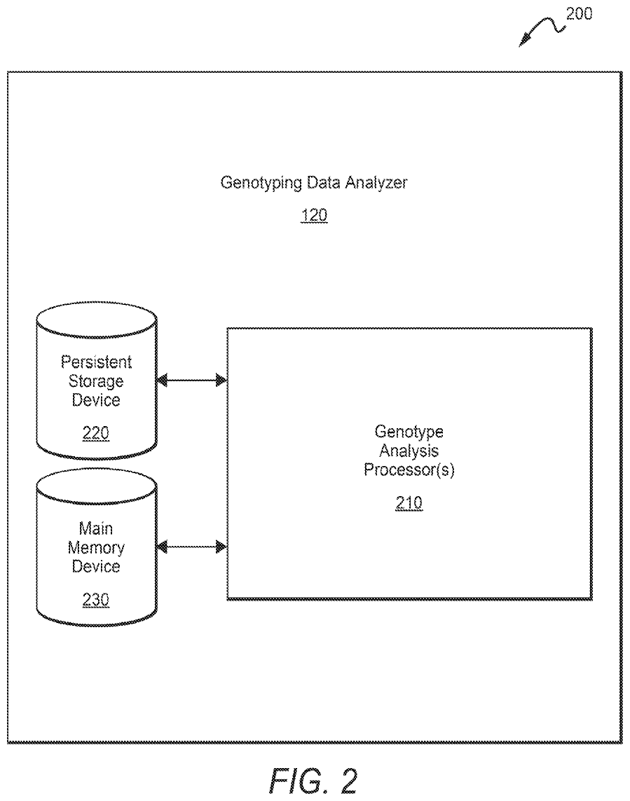

FIG. 2 illustrates a block diagram 200 of genotyping data analyzer 120 according to embodiments. Genotyping data analyzer 120 may comprise various localized or distributed elements for genotyping a gene sequence, including one or more processors 210 of at least one computing device, persistent storage device 220, and main memory device 230. In an embodiment, genotyping data analyzer 120 may be configured to receive or obtain first genotyping call data representing a gene sequence of interest, i.e., a query gene sequence, which may be stored in either one or both of persistent storage device 220 and main memory device 230. Further, persistent storage device 220, and main memory device 230 are configured to store one or more instructions, which, when executed by the one or more processors 210, cause the one or more processors to perform one or more functions. In various embodiments, the one or more functions may include: assigning a numerical score to each of a plurality of allele calls of second genotyping call data by matching the first genotyping call data with second genotyping call data, the second genotyping call data representing a plurality of candidate gene sequences; determining a match score for each of the plurality of candidate gene sequences based on the numerical score assigned to each of the plurality of allele calls of the second genotyping call data; and making a genotyping call for the query gene sequence based on a highest match score from among the match score determined for each of the plurality of candidate gene sequences.

It should be noted that the elements in FIG. 2, and the various functions attributed to each of the elements, while exemplary, are described as such solely for the purposes of ease of understanding. One skilled in the art will appreciate that one or more of the functions ascribed to the various elements may be performed by any one of the other elements, and/or by an element (not shown) configured to perform a combination of the various functions. Therefore, it should be noted that any language directed to genotyping data analyzer 120, one or more processors 210, persistent storage device 220 and main memory device 230 should be read to include any suitable combination of computing devices and/or computer-based network platforms, including servers, interfaces, systems, databases, agents, peers, engines, controllers, modules, or other types of computing devices operating individually or collectively to perform the functions ascribed to the various elements. Further, one skilled in the art will appreciate that one or more of the functions of genotyping data analyzer 120 of FIG. 2 described herein may be performed within the context of a client-server relationship, such as by one or more servers, one or more client devices (e.g., one or more user devices) and/or by a combination of one or more servers and client devices, including by any combination of the elements (PCR apparatus 100, capillary electrophoresis apparatus 110, and genotyping data analyzer 120) of system 10.

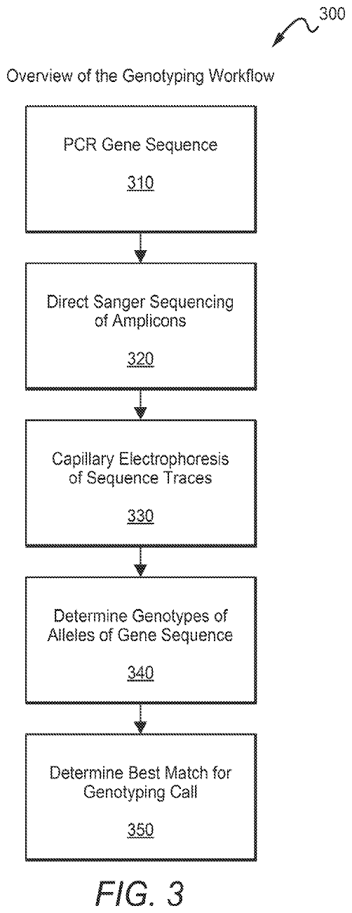

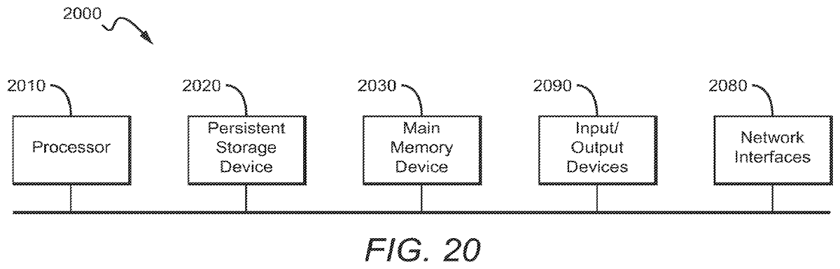

FIG. 3 illustrates an exemplary functional block diagram of a genotyping workflow process 300 that can be used to genotype a query gene sequence using a system comprising a PCR apparatus, capillary electrophoresis apparatus, and genotyping data analyzer (e.g., PCR apparatus 100, capillary electrophoresis apparatus 110, and genotyping data analyzer 120 of system 10). The blocks illustrated in FIG. 3 represent processes and/or methods that can be implemented and/or executed by one or more computing elements described below with reference to FIG. 20 (e.g., by one or more computing devices). The computing elements can be standalone computing elements, networked computing elements, distributed computing elements, and/or embedded computing elements. For example, the computing elements can be integrated with PCR apparatus 100, capillary electrophoresis apparatus 110, genotyping data analyzer 120, an onsite standalone computing device, a cloud-based computing device, or a combination thereof.

In general, genotyping workflow process 300 comprises a PCR, Sanger sequencing, and capillary electrophoresis workflow. For example, in various embodiments, process 300 can include DNA template preparation (e.g., by PCR); cycle sequencing; purification of extension products after cycle sequencing; capillary electrophoresis; and data analysis (e.g., applying analysis profiles, running analyses, and allowing a review of data). However, one skilled in the art will recognize that an implementation of genotyping workflow process 300 may optionally include various process steps in addition to those described here, or not include various ones of the process steps described here, and that genotyping workflow process 300 is a high-level representation of a workflow that may be applied to implement the various embodiments described herein.

With reference to FIG. 3, genotyping workflow process 300 begins at process step 310, where a query gene sequence is PCR amplified, in preparation for Sanger sequencing, using a PCR apparatus (e.g., PCR apparatus 100). In some embodiments, PCR amplification may include digital PCR (dPCR) processes. For example, as mentioned above, digital PCR may use a solution including a relatively small number of a target analyte, e.g., a polynucleotide or nucleotide sequence template DNA (or RNA), fluorescence-quencher probes, primers, and a PCR master mix comprising DNA polymerase and reaction buffers at optimal concentrations. The solution may be partitioned into a large number of small test samples, e.g., tens of thousands of microchambers disposed within a microfluidic array plate, and thermal cycling may be subsequently performed with respect to array of partitions using a PCR apparatus (e.g., PCR apparatus 100).

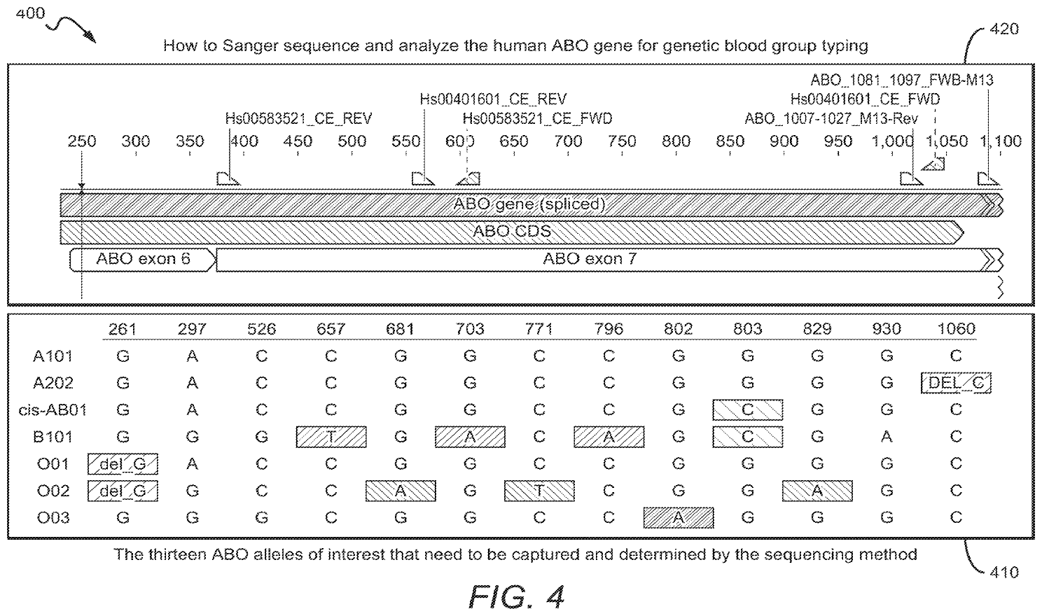

In the use case of determining a specimen's (diploid) blood group genotype, a large portion (e.g., 992 bases=82.4%) of the 1065 bp coding sequence of the human ABO gene may be PCR amplified in preparation for Sanger sequencing. FIG. 4 illustrates a diagram of regions of interest for a human ABO gene sequence and genotyping call data representing the seven major blood group genotypes determined by the allele variants in a human ABO gene sequence. In diagram 400, it is shown that the seven major blood group genotypes 410 are determined by the 13 allele variants in exons 6 and 7 in the human ABO gene sequence, shown in sequence diagram 420. While some variants are specific for a particular blood type, other variants are shared between blood groups. Based on this information, the 13 allele variants in exons 6 and 7 in the human ABO gene sequence may comprise a region of interest for amplification and Sanger sequencing.

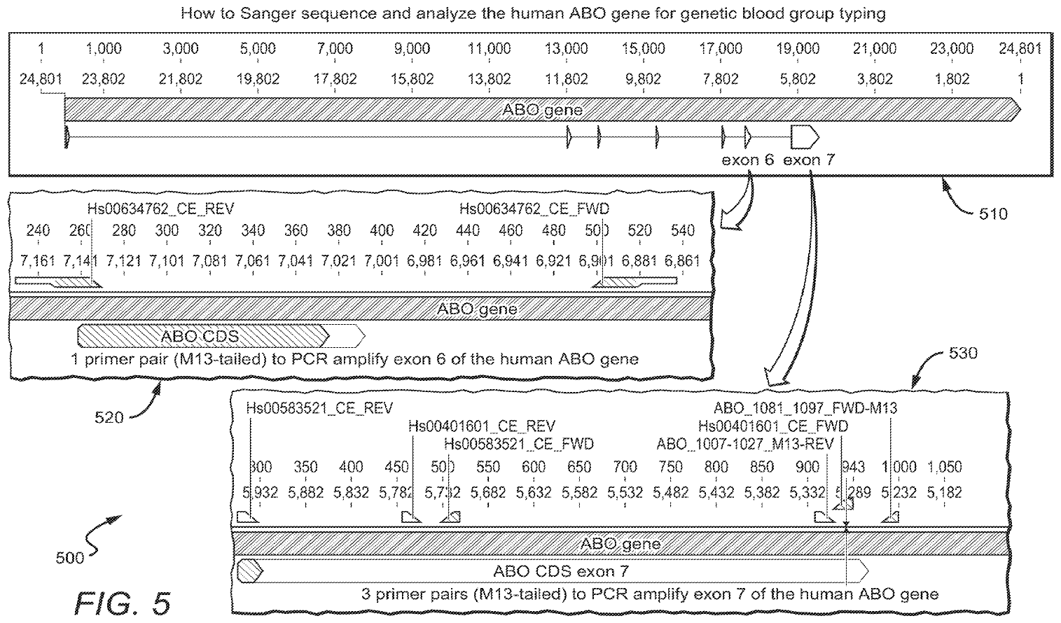

In some embodiments, various amplicons are applied to capture a region of interest in a gene sequence for PCR amplification and subsequent Sanger sequencing. FIG. 5 illustrates a diagram of genomic loci and primers used for amplifying alleles of interest in a query gene sequence. In some embodiments, an amplicon/primer pair design may be selected or determined to amplify alleles of interest in, e.g., exons 6 and 7 of the human ABO gene 510. For example, as shown in FIG. 5, one PCR amplicon may be selected for exon 6 to cover 271 bases, and three amplicons may be selected for exon 7 to cover 721 bases. The four PCR amplicons may then be “tailed” (by the PCR process) with the M13 forward primer sequence at the 5′ end and the M13-reverse primer sequence at the 3′ end (vice versa for ABO_amplicon 4; as described below). The M13 tails may serve as primer binding sites for subsequent Sanger sequencing reactions (e.g., eight BigDyeR Direct Sanger sequencing reactions; four in the forward direction and four in the reverse direction).

In some embodiments, primer pair 520 may be selected to PCR amplify exon 6 of the human ABO gene in accordance with the following specifications, where the underlined sequence indicates the ABO gene-specific part:

ABO primer pair 1: Hs00634762

### Exon 6:

### Primer Chromosome Location

Chr.9:133257282-133257553 on Build GRCh38

### Amplicon Length 272 bp

Forward Primer with M13 Tail 5′

(SEQ ID NO. 1)
TGTAAAACGACGGCCAGTCATCTACCCTCTGGGAGGACA 3′

Reverse Primer with M13 Tail 5′

(SEQ ID NO. 2)
CAGGAAACAGCTATGACCTCCATGTGCAGTAGGAAGGATG 3′

Likewise, in some embodiments, primer pair 530 may be selected to PCR amplify exon 7 in accordance with the following specifications, where the underlined sequence indicates the ABO gene-specific part:

ABO_primer pair 2: Hs00583521

### Exon 7:

### Primer Chromosome Location

Chr.9:133256114-133256355 on Build GRCh38

### Amplicon Length 242 bp

Forward Primer with M13 Tail 5′

(SEQ ID NO. 3)
TGTAAAACGACGGCCAGTTAATCCACCTCGCTGAGGAAG 3′

Reverse Primer with M13 Tail 5′

(SEQ ID NO. 4)
CAGGAAACAGCTATGACCTACGTGGCTTTCCTGAAGCTG 3′

ABO_primer pair 3: Hs00401601

### Exon 7:

### Primer Chromosome Location

Chr.9:133255684-133256175 on Build GRCh38

### Amplicon Length 492 bp

Forward Primer with M13 Tail 5′

(SEQ ID NO. 5)
TGTAAAACGACGGCCAGTCTGGTGGTTCTTGGGCACCG 3′

Reverse Primer with M13 Tail 5′

(SEQ ID NO. 6)
CAGGAAACAGCTATGACCATGCGCCGCATGGAGATGAT 3′

ABO_primer pair 4: ABO_1081-1097_FWD-M13 and ABO_1007-1027_REV-M13
Thermofisher.com order #68885006
Forward primer: ABO_1007-1027_M13-REV (target seq binds to lower strand)

Sequence:
(SEQ ID NO. 7)
5′ CAGGAAACAGCTATGACCGAGGAAGCTGAGGTTCACTGC 3′

Thermofisher.com order #67509220
Reverse primer: ABO_1081-1097_M13-FWD (target seq binds to upper strand)

Sequence:
(SEQ ID NO. 8)
5′ TGTAAAACGACGGCCAGTCCGGCAGCCCTCCCAGA 3′

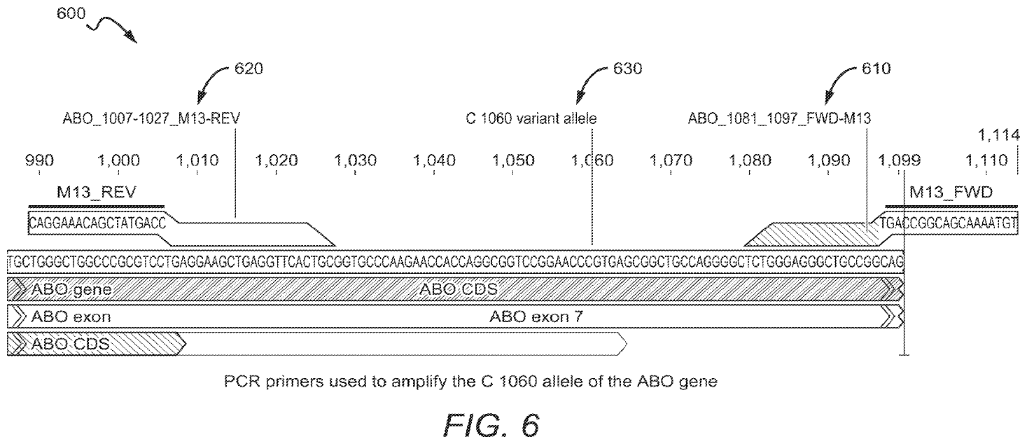

FIG. 6 illustrates a diagram 600 of PCR primers applied to amplify, using PCR, alleles of interest in a query gene sequence. For example, in accordance with ABO_primer pair 4 discussed above with reference to FIG. 5, forward PCR primer 610 (ABO_1081-1097_FWD-M13) and reverse PCR primer 620 (ABO_1007-1027_REV-M13) may be applied to amplify the C 1060 allele 630 of the query gene sequence. To genotype the deletion “C” base variant at position 1060 of the ABO gene reliably without compromising the sequencing quality of upstream sequences, this dedicated primer pair/amplicon may be designed for sequencing this particular allele.

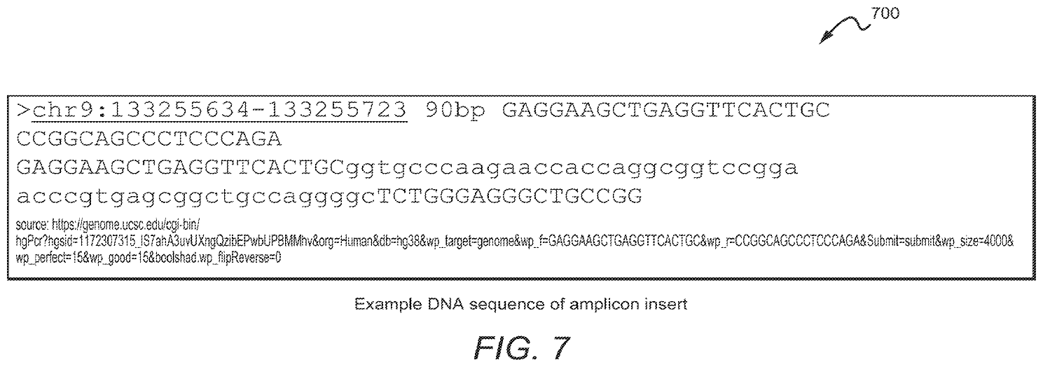

FIG. 7 illustrates the DNA sequence 700 of the amplicon insert applied to amplify the C 1060 allele 630. In a heterozygous deletion, mixed bases are expected in the Sanger sequencing traces 3′ after the deleted nucleotide when using this dedicated primer pair/amplicon design. In the case of a homozygous deletion (indicative of a homozygous A2/A2 genotype), an apparent lack of a “C” base in the alignment of the specimen traces with a reference sequence are expected.

One skilled in the art will recognize that other PCR primer/amplicon designs may be selected for PCR amplifying and sequencing particular alleles of interest in a query gene sequence. One skilled in the art will further recognize that such PCR primer/amplicon designs may account for a variety of considerations, including, e.g., a desire to genotype heterozygous and/or homozygous deletions reliably, to genotype without compromising the sequencing quality of upstream sequences, or the like.

In some embodiments, a composition and kit comprising one or more of sequences of SEQ ID NOs. 1-8 are provided. In some embodiments, the composition and kit are designed to genotype a gene or genes of interest. In some embodiments, the sequence or sequences from the composition and kit may be a derivative of any sequence or sequences of SEQ ID NOs. 1-8. In some embodiments, the derivative sequence refers to a sequence having a sequence identity of about or at least 50%, about or at least 55%, about or at least 60%, about or at least 65%, about or at least 70%, about or at least 75%, about or at least 80%, about or at least 85%, about or at least 86%, about or at least 87%, about or at least 88%, about or at least 89%, about or at least 90%, about or at least 91%, about or at least 92%, about or at least 93%, about or at least 94%, about or at least 95%, about or at least 96%, about or at least 97%, about or at least 98%, about or at least 99%, or about 100% to any sequence or sequences of SEQ ID NOs. 1-8. In some embodiments, the derivative sequence refers to a sequence having 1 base, 2 bases, 3 bases, 4 bases, 5 bases, 6 bases, 7 bases, 8bases, 9 bases, 10 bases, 11 bases, 12 bases, 13 bases, 14 bases, 15 bases, 16 bases, 17 bases, 18 bases, 19 bases or 20 bases different from any sequence or sequences of SEQ ID NOs. 1-8. In some embodiments, the composition and kit contain SEQ ID NOs. 1-2 or any derivative of SEQ ID NOs. 1-2. In some embodiments, the composition and kit contain SEQ ID NOs. 3-4 or any derivative of SEQ ID NOs. 3-4. In some embodiments, the composition and kit contain SEQ ID NOs. 5-6 or any derivative of SEQ ID NOs. 5-6. In some embodiments, the composition and kit contain SEQ ID NOs. 7-8 or any derivative of SEQ ID NOs. 7-8. In some embodiments, the composition and kit contain SEQ ID NOs. 7-8 or any derivative of SEQ ID NOs. 7-8 and further contain any one or more of SEQ ID NOs. 1-6. In some embodiments, the kit may further comprise a DNA polymerase and additional components (e.g., a buffer, dNTPs, MgC12, enhancers and stabilizers in a buffer) necessary or desirable for gene amplification. Also provided according to some embodiments is a method of genotyping a target sequence using any of the compositions or kits as disclosed herein.

With reference back to FIG. 3, and continuing with process step 310, a PCR amplification of the query gene sequence, e.g., with four PCR reactions corresponding to the ABO_PCR primer pairs 1-4 (referenced in FIG. 5 above), is performed. For example, each of the four ABO_PCR reactions for an individual sample may be set up in a PCR plate (such as that sold under MicroAmp™ PCR plate) as follows:
5 μl BigDyeR Direct PCR master mix (part of BigDye® Direct Cycle Sequencing Kit, 1000 reactions SKU #4458688)
3.5 μl Dist. H20
0.5 μl Specific ABO_PCR primer pair (10 μM ea; M13-tailed)
0.6-1 μl Genomic DNA of the specimen (DNA concentrations are variable, typically ranging 1-10 ng)

Note that, usually, the four ABO_PCR reactions are set up as four complete premixes for multiple samples (e.g., 10), leaving out the gDNAs.

The plate is covered with an optical adhesive film (such as MicroAmp™ Optical Adhesive Film sold by Thermo Fisher Scientific), and then vortexed briefly for 2-3 seconds. The plate is centrifuged briefly (e.g., 10-20 seconds at 500-1000 rpm) in a plate centrifuge to force the reaction liquid to the bottom of the well.

Further, PCR may be performed using a thermal cycler, e.g., such as sold under the name Applied Biosystems ProFlex™ PCR System, using the following cycling parameters shown in Table 1 below:

TABLE 1

| Stage | Temperature | Time | |
|---|---|---|---|
| Hold | 95° C. | 6 | min |
| Cycle | 96° C. | 3 | sec |
| (35 cycles) | 62° C. | 15 | sec |
| | 68° C. | 30 | sec |
| Hold | 72° C. | 2 | min |

| | | |
|---|---|---|
| Hold | 4° C. | Until stop |

Referring to FIG. 3, in process step 320, the query gene sequence is sequenced in the forward direction and the reverse direction using fluorescent dye-terminator Sanger sequencing. For example, following PCR process step 310, two batches of sequencing mixes may be set up: one forward and one reverse sequencing mix. In some embodiments, it may be desirable to Sanger sequence a plurality of specimens (e.g., for genomic DNA specimens). For example, to Sanger sequence 10 specimens each having 4 PCR amplicons, the following batches may be required: 4 (i.e., ABO amplicons 1-4)×10 (i.e. # of specimen)=40 +5% overage=42 forward sequencing reactions and 42 reverse sequencing reactions.

Continuing with the example described above, an individual Sanger sequencing reaction may set up as follows:

In practice, a premix of forward and a premix of reverse sequencing reagent is prepared for all samples+overage; therefore, an appropriate multiple is used for the amounts shown below for a single reaction.
6 μl Dist. H₂0
1.5 μl BigDye® Terminator v1.1 & v3.1 5× Sequencing Buffer SKU #4336697 (Thermo Fisher Scientific)
1 μl BigDyeR Direct Sequencing Master Mix (part of BigDyeR Direct Cycle Sequencing Kit, 1000 reactions SKU #4458688) (Thermo Fisher Scientific)
0.5 μl BigDyeR Direct M13 Fwd Primer or BigDye® Direct M13 Rev Primer (both items are part of the BigDyeR Direct Cycle Sequencing Kit, 1000 reactions SKU #4458688)
Total 9 μl

9 μl of sequencing mix is dispensed into wells of a 96-well skirted sequencing plate (such as sold under MicroAmp™ plate by Thermo Fisher Scientific). 1 μl of PCR product is added. The plate is covered with an optical adhesive film (such as MicroAmp™ Optical Adhesive Film sold by Thermo Fisher Scientific), and briefly vortexed for 2-3 seconds. The plate is then briefly centrifuged (e.g., 10-20 seconds at 500-1000 rpm) in a plate centrifuge to force the reaction liquid to the bottom of the plate well.

As discussed above, a Sanger sequencing workflow may include cycle sequencing, and purification of extension products after cycle sequencing. For example, the MicroAmp plate referenced above may be placed in a thermal cycler instrument, such as sold under Applied Biosystems ProFlex™ PCR System, for BigDye® Direct cycle sequencing using the following default settings on the thermal cycler instrument shown in Table 2 below:

TABLE 2

| Stage | Temperature | Time | |
|---|---|---|---|
| Hold | 37° C. | 15 | min |
| Hold | 80° C. | 2 | min |
| Hold | 96° C. | 1 | min |
| Cycle | 96° C. | 10 | sec |
| (25 cycles) | 50° C. | 5 | sec |
| | 60° C. | 75 | sec |

| | | |
|---|---|---|
| Hold | 4° C. | Until stop |

The finished cycle sequencing reactions may then be purified from unincorporated fluorescent dye-terminator nucleotides, before capillary electrophoresis. For example, in an exemplary embodiment, the cycle sequencing reactions may be purified using the BigDye® Xterminator™ purification kit (BDX) reagent from Applied Biosystems SKU #4376484.

With reference to FIG. 3, in process step 330, capillary electrophoresis may be used to separate the extension products resulting from the Sanger sequencing of the sequence traces. For example, in an exemplary embodiment after purification, the finished cycle sequencing reactions may be subjected to capillary electrophoresis sequencing (CE) on an Applied Biosystems™ 3500×L Genetic Analyzer or Applied Biosystems™ SeqStudio® Genetic Analyzer.

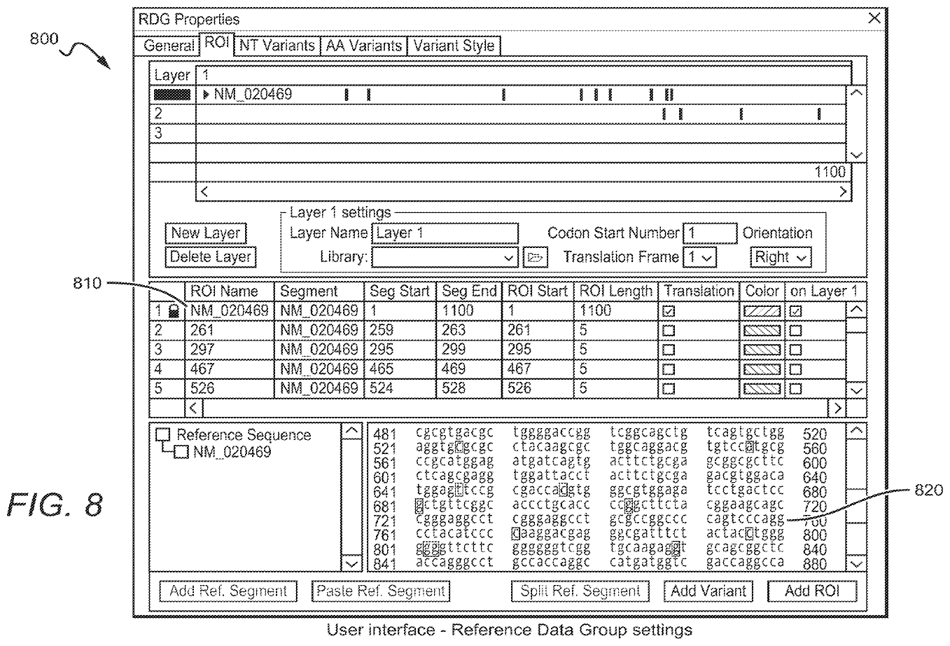

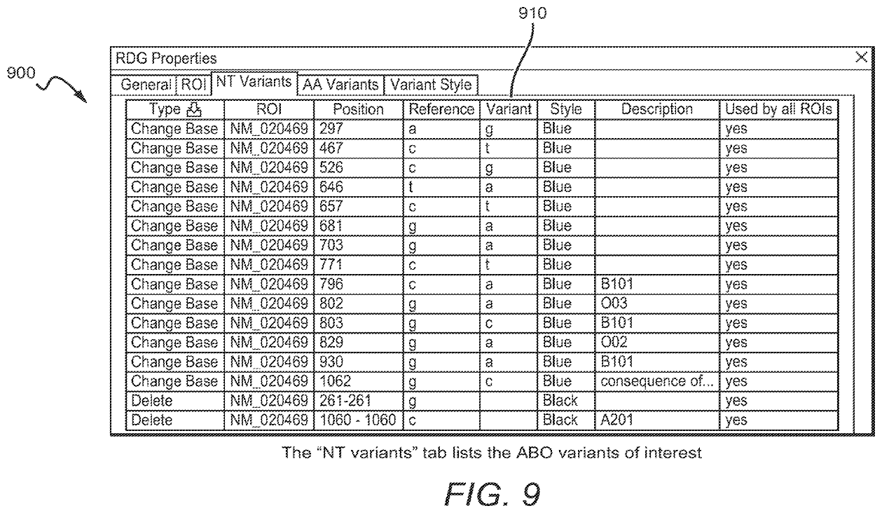

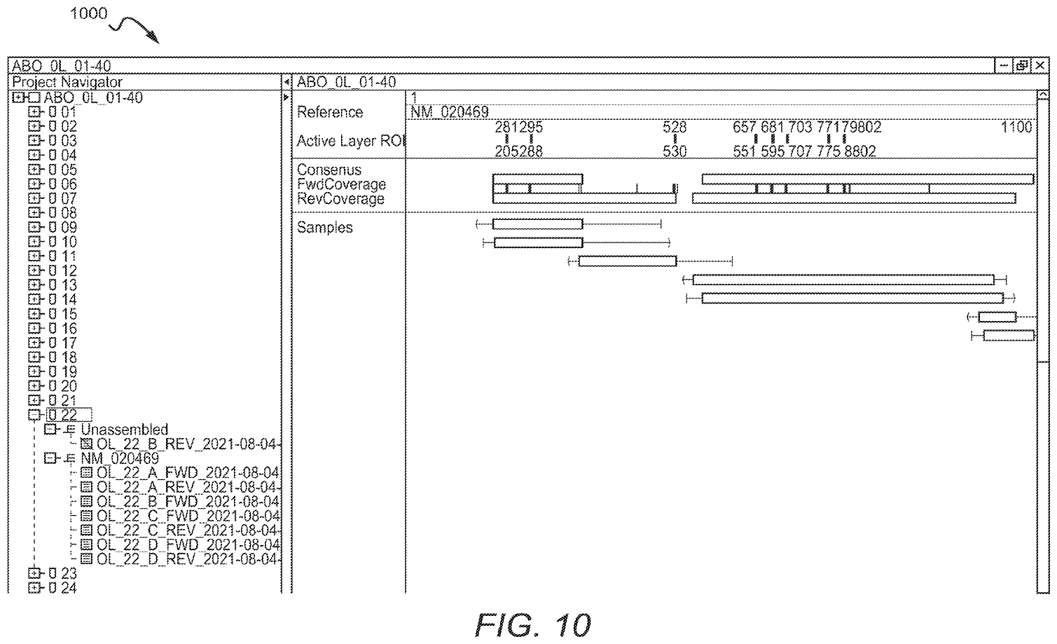

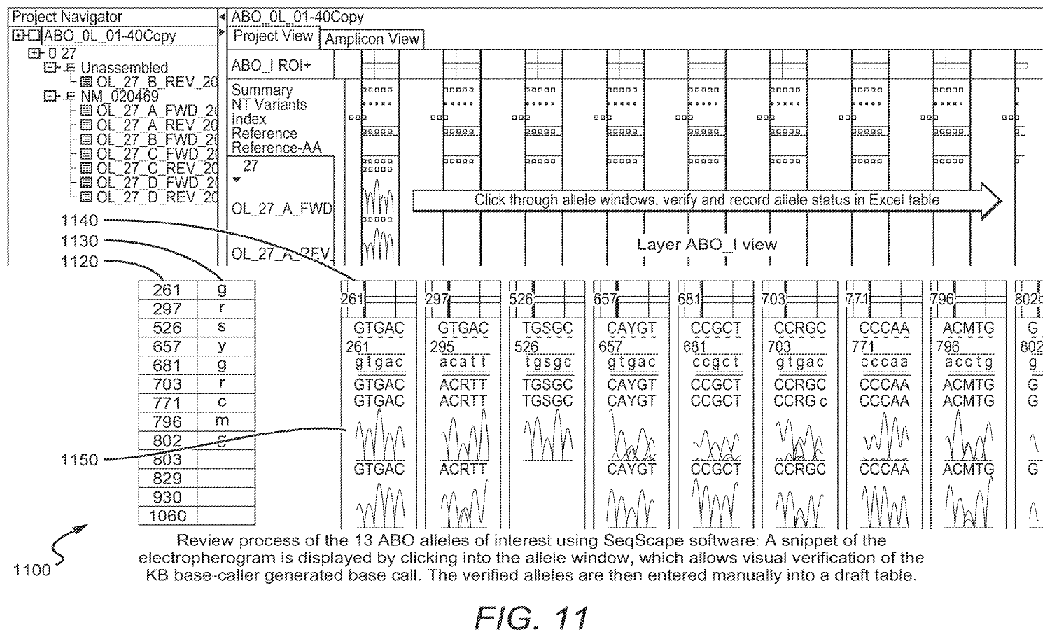

With reference to FIG. 3, in process step 340, a genotyping call is made for each of a plurality of alleles of the query gene sequence based on the Sanger-based DNA sequencing data. FIG. 8 illustrates an interface display of data settings for a reference gene sequence 810, 820. Further, with reference to FIG. 9, in some embodiments RDG file 800 may include a dedicated tab 900 for visualization of variants for alleles of interest in a query gene sequence. For example, genotyping data for an allele variant of interest 910 may be configured for visualization of allele positions, allele calls, and other genotyping data. For example, visual reports and charts of the results of the Sanger-based DNA sequencing data can be generated and displayed to a user. In some embodiments, a “reference data group” (or RDG) file 800 may be generated for a customized genotyping analysis (e.g., an ABO genotyping analysis) of the.ab1 sequence output files for the query gene sequence. For example, the resulting sequencing data files (n=8 per specimen) in .ab1 file format may represent allele call data that can be analyzed to make a genotyping call for the query gene sequence. In FIG. 10, trace files are aligned to a reference sequence of the ABO gene (NM020469) 810, 820. In FIG. 11, the thirteen alleles of interest that determine the major blood group genotypes are presented in a “review mask” 1100 to allow for visual examination and verification of proper base calls. In some embodiments, the trace files may be structured in two or more layers 1110 for better clarity.

With reference to FIG. 10, an interface display 1000 of an assembly overview of query gene sequence traces to a reference gene sequence 810, 820 is shown. In some embodiments, an optional step can be performed to verify sequence trace assembly and alignment. For example, interface display 1000 includes an assembly overview of sequence traces to the ABO reference gene sequence (NM020469). Note that in the shown sample set of eight traces, seven traces assembled correctly, whereas one file could not be assembled because of poor data quality (which is acceptable since the other strand is of high quality). In some embodiments, analysis software may be configured to automatically re-analyze the input data (i.e., the base calls) and determined deviations from the reference sequence.

With reference to FIG. 11, an interface display 1100 corresponding to alleles of interest obtained after a KBTM Basecaller generated base call is shown. For example, visual reports and charts of the results of base call data can be generated and displayed to a user. In some embodiments, a review process of base calls for the 13 ABO alleles of interest 1120 can be visualized, e.g., using Applied Biosystems™ SeqScape™ analysis software. For example, an electropherogram snippet 1150 may be displayed by clicking into an allele window 1140, which can allow for visual verification of the KB™ Basecaller generated base call. In some embodiments, the alleles of interest 1120 may be visualized in two or more layers, e.g., ABO_I and ABO_II, to separate the review of the alleles for better clarity.

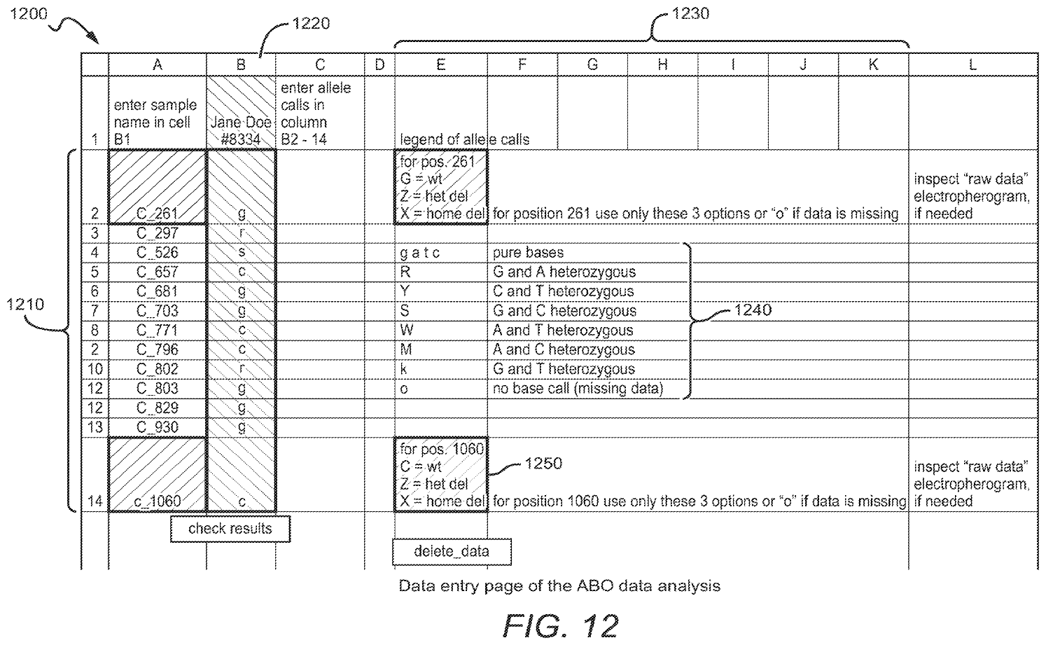

FIG. 12 illustrates an interface display 1200 of allele call data 1210 corresponding to alleles of interest in a query gene sequence. In some embodiments, allele call data 1210, e.g., for a subset may be represented. For example, the first genotyping call data 1210 can comprise a subset of genotyping call data, where the subset of the genotyping call data corresponds to ABO alleles of interest 1220 used for determining major ABO blood types. A legend of allele calls 1230 may be used to interpret the allele call data 1210. For example, the allele call data 1210 may comprise International Union of Pure and Applied Chemistry (IUPAC) codes 1240, and a heterozygous or homozygous deletion code (e.g., “Z” and “X”, respectively) 1250.

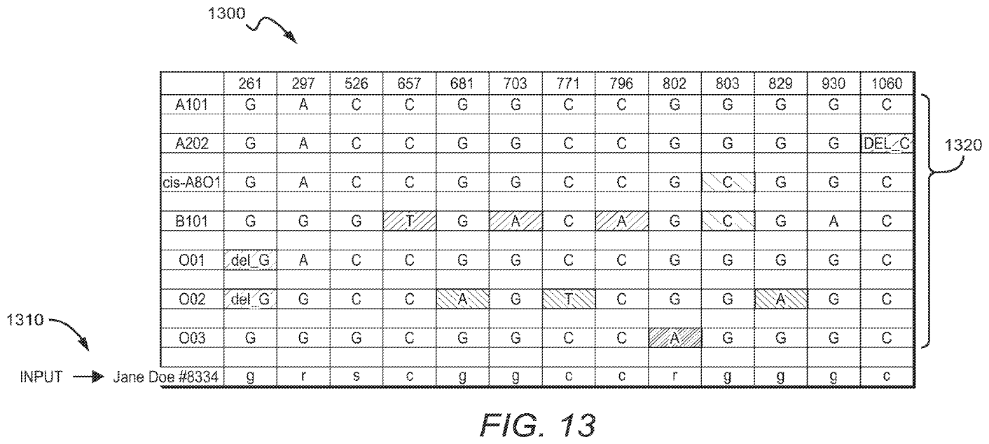

FIG. 13 illustrates an interface display 1300 of allele call data corresponding to alleles of interest in a query gene sequence. In FIG. 13, the first genotyping data 1310 of column B1-14 (FIG. 12) representing the query gene sequence is displayed with genotyping data 1320 representing a plurality of candidate gene sequences, e.g., corresponding to one or more known phenotypes. For example, the one or more known phenotypes may comprise ABO phenotypes, as shown.

With reference to FIG. 3, in process step 350, a genotyping call is made for the query gene sequence based on a highest match score from among match scores determined for each of the plurality of candidate gene sequences. For example, a highest-scoring match may indicate the diploid ABO blood type genotype for a query gene sequence.

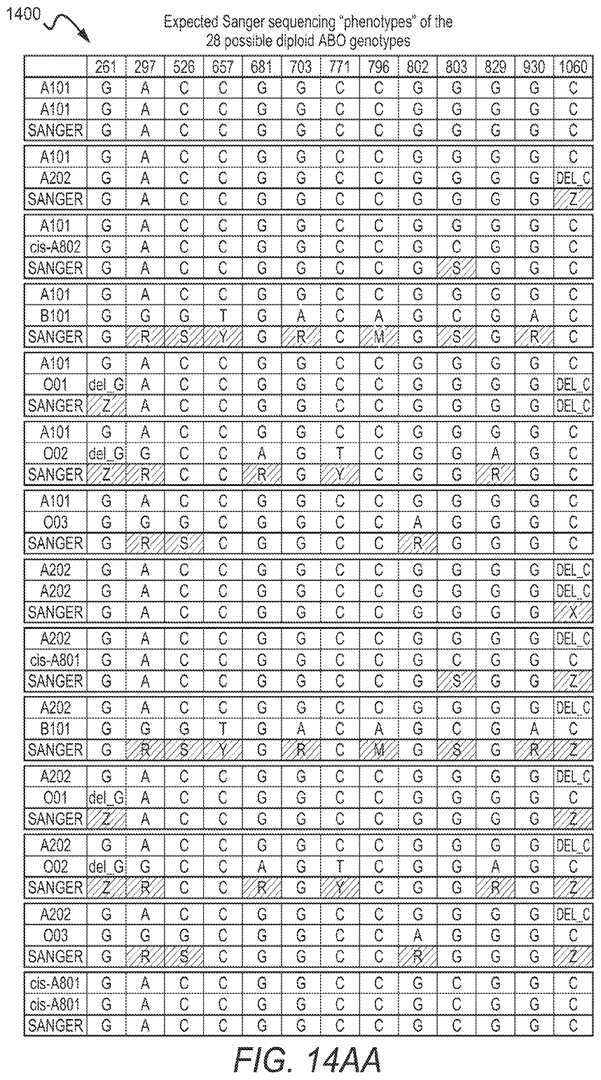

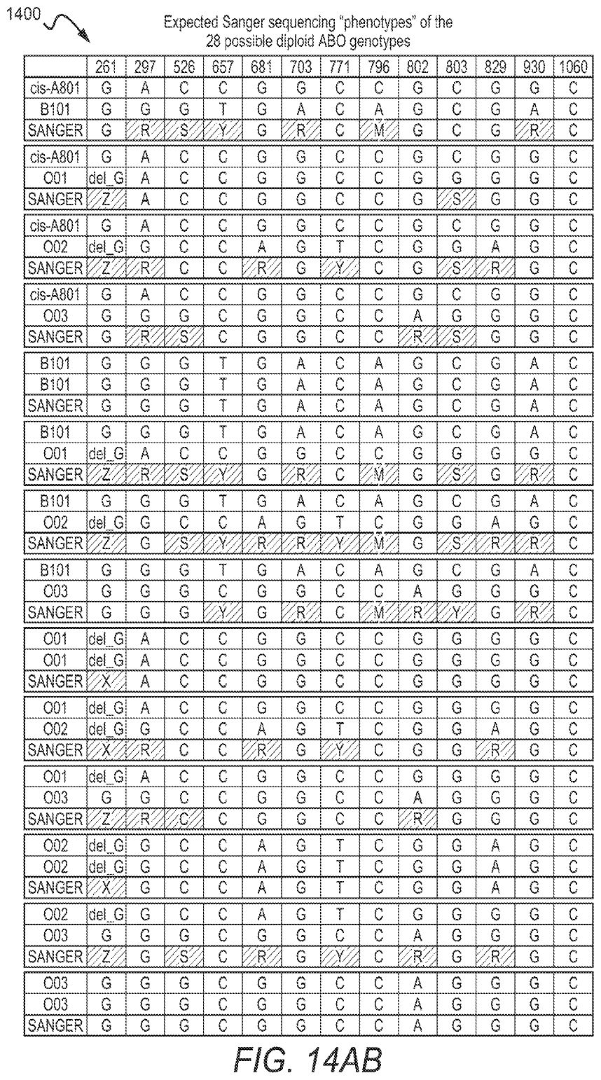

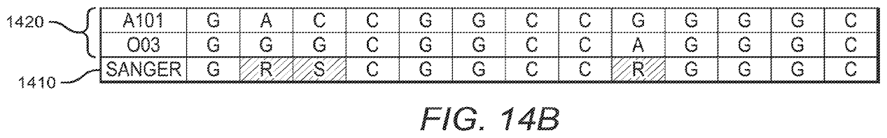

FIG. 14A illustrates a genotyping call data look-up table 1400 according to embodiments. Data look-up table 1400 represents a plurality of candidate gene sequences. In some embodiments, the plurality of candidate gene sequences comprises the 28 possible “Sanger phenotype” results of the diploid combinations of the different blood group genotypes. For example, look-up table 1400 lists the 28 possible diploid pairs of genotypes given the seven major blood types to be determined. With reference to FIG. 14B, the row “Sanger” 1410 under each diploid pair indicates how the combination of the parental genotypes 1420 will present in a fluorescent dye-terminator Sanger sequencing trace. For example, a heterozygous allele of “A” and “G” will present as a “R” base call, and a heterozygous “C” and “T” as “Y” in the electropherogram. In some embodiments, a heterozygous or homozygous deletion (e.g., at alleles 261 or 1060) may be denoted by codes, e.g., “z” or “x”, respectively. While the “z” and “x” codes are not official IUPAC codes, they may be advantageous for robust data processing since a deletion may perturb base calling but is readily detectable at the data review stage.

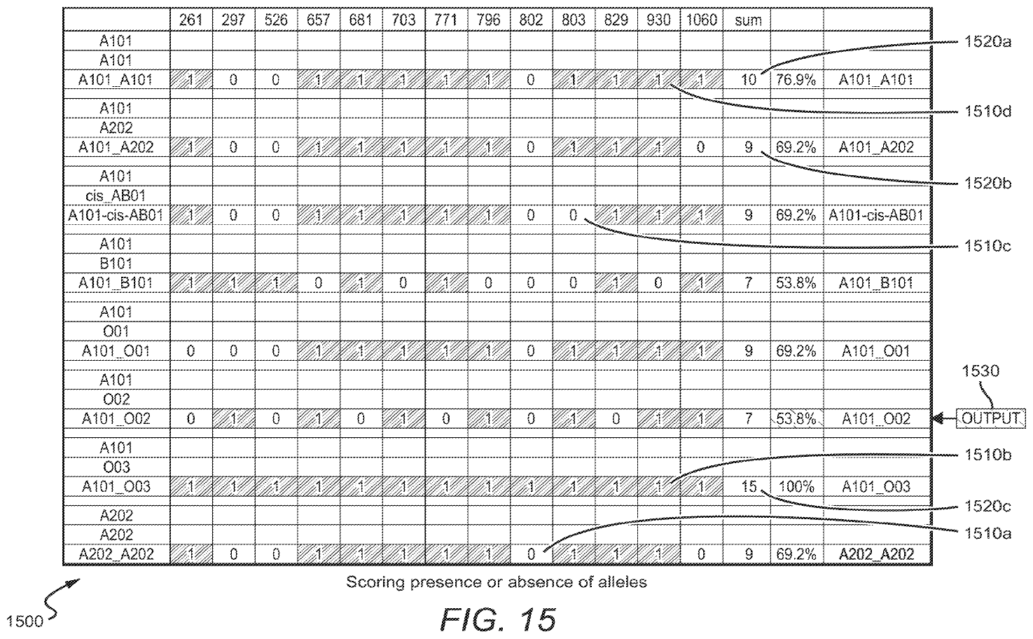

FIG. 15 illustrates an interface display 1500 of numerical scores assigned to each of a plurality of allele calls of second genotyping call data representing a plurality of candidate gene sequences and a match score determined for each of the plurality of candidate gene sequences. For example, visual reports and charts of the results of the matching can be generated and displayed to a user. In some embodiments, the observed/recognized genotype of a specimen (a query gene sequence) may be compared with expected “phenotype” results obtained based on Sanger sequencing data corresponding to the 28 possible diploid combinations corresponding to different blood group genotypes. For example, the process may include assigning a numerical score, e.g., numerical scores 1510a-d, to each of a plurality of allele calls of genotyping call data representing the plurality of candidate gene sequences by matching the genotyping call data representing the query gene sequence with the genotyping call data representing the plurality of candidate gene sequences. The process may further include determining a match score, e.g., match scores 1520a-c, for each of the plurality of candidate gene sequences based on the numerical score assigned to each of the plurality of allele calls of the genotyping call data representing the plurality of candidate gene sequences; and making a genotyping call, e.g., genotyping call 1530, for the query gene sequence based on a highest match score from among the match scores determined for each of the plurality of candidate gene sequences. In some embodiments, the scoring may be done by a logical presence/absence test. For example, if there is a “G” at allele position 261 in the genotyping call data representing the query gene sequence and this allele with the value “G” matches genotyping call data representing one of the candidate gene sequences at position 261 in the look-up table (e.g., in FIG. 14), it will be scored as “1”. If there is no “G” in the genotyping call data representing one of the candidate gene sequences at position 261, then it will be scored as “0” (i.e., zero).

The same process is applied for all 13 ABO alleles of interest. The individual scores are summed up and the highest score 1520c (here shown as “13”=100%) indicates the diploid blood group genotype.

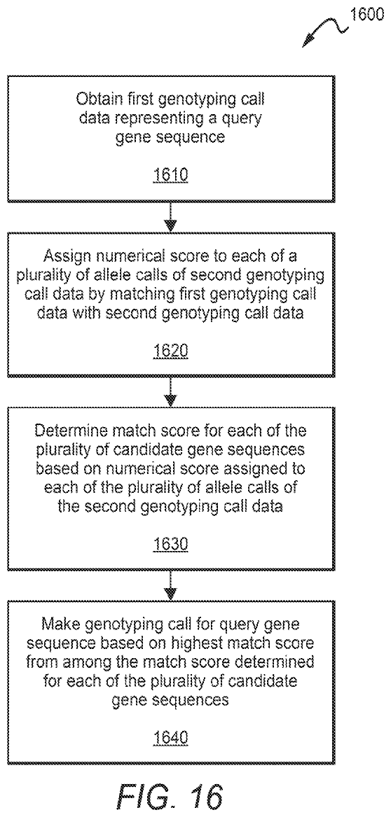

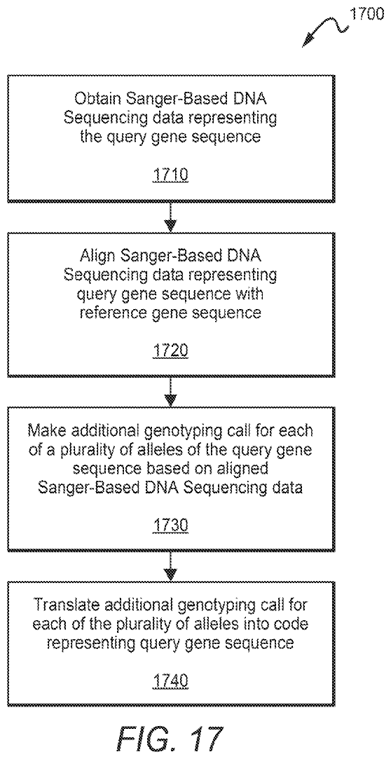

With reference to FIG. 16, a method 1600 is provided. Method 1600 is for genotyping a gene sequence using a system (e.g., including PCR apparatus 100, capillary electrophoresis apparatus 110, and genotyping data analyzer 120 in FIG. 1) configured to analyze a gene sequence according to example embodiments. In one embodiment, method 1600 begins with step 1610, during which first genotyping call data representing a query gene sequence is received or obtained. For example, FIG. 17 is a flowchart illustrating a method 1700 for obtaining the first genotyping call data. In some embodiments, obtaining the first genotyping call data may begin with step 1710 of obtaining Sanger-based DNA sequencing data representing the query gene sequence as described above with respect to FIGS. 3-7. At step 1720, the Sanger-based DNA sequencing data representing the query gene sequence is aligned with a reference gene sequence as described above with respect to FIG. 8. In step 1730, an additional genotyping call is made for each of a plurality of alleles of the query gene sequence based on the aligned Sanger-based DNA sequencing data. In some embodiments, the additional genotyping call for each of the plurality of alleles may be translated into a code representing the query gene sequence as described above with respect to FIGS. 11-13 in step 1740. For example, the code may comprise an International Union of Pure and Applied Chemistry (IUPAC) code, and a heterozygous or homozygous deletion code.

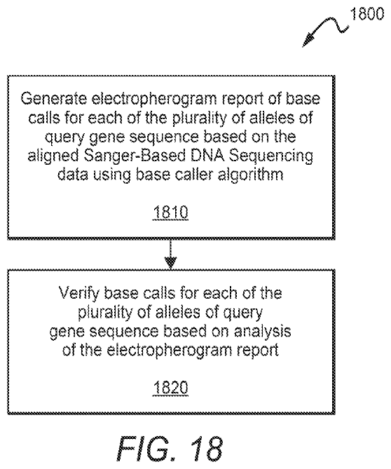

FIG. 18 is a flowchart illustrating a method 1800 for making the additional genotyping call in accordance with various embodiments. With reference to FIG. 18 in step 1810, making the additional genotyping call may include generating an electropherogram report of base calls for each of the plurality of alleles of the query gene sequence based on the aligned Sanger-based DNA sequencing data using at least one base caller algorithm. For example, the at least one base caller algorithm may be KBTM Basecaller. In step 1820, the base calls may be verified for each of the plurality of alleles of the query gene sequence based on an analysis of the electropherogram report.

With reference back to FIG. 16, in step 1620, a numerical score is assigned to each of a plurality of allele calls of second genotyping call data by matching the first genotyping call data with the second genotyping call data, the second genotyping call data representing a plurality of candidate gene sequences. In some embodiments, as described above with reference to FIG. 15, the method may comprise assigning a first numerical value of “1” if there is a positive match between an allele call of the first genotyping call data and a corresponding allele call of the second genotyping call data, and a second numerical value of “0” if there is a non-positive match between the allele call of the first genotyping call data and the corresponding allele call of the second genotyping call data.

In some embodiments, the method may comprise generating a look-up table (as described above with reference to FIGS. 14A-14B) comprising the second genotyping call data, where the look-up table comprises a list of codes representing each of the plurality of candidate gene sequences, and where the list of codes comprises International Union of Pure and Applied Chemistry (IUPAC) codes, and a heterozygous or homozygous deletion code. For example, the matching (with reference to FIG. 16, in step 1620) of the first genotyping call data with the second genotyping call data may further include using at least one find operation to query the look-up table using the first genotyping call data.

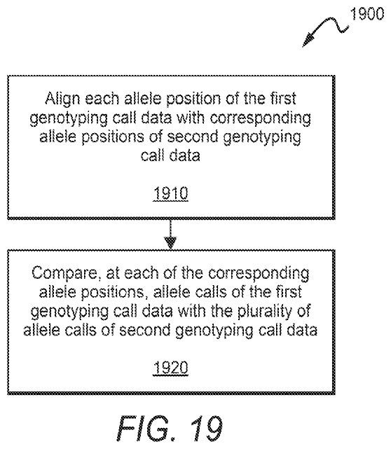

FIG. 19 is a flowchart illustrating a method 1900 for matching of the first genotyping call data with the second genotyping call data in accordance with various embodiments. In step 1910, the matching (with reference to FIG. 16, in step 1620) of the first genotyping call data with the second genotyping call data may further include aligning each allele position of the first genotyping call data with corresponding allele positions of the second genotyping call data. In step 1920, at each of the corresponding allele positions, allele calls of the first genotyping call data are compared with the plurality of allele calls of the second genotyping call data for an assignment of a numerical score to each of a plurality of allele calls of second genotyping call data.

With reference back to FIG. 16, in step 1630 a match score is determined for each of the plurality of candidate gene sequences based on the numerical score assigned to each of the plurality of allele calls of the second genotyping call data. In an embodiment, the determining of the match score for each of the plurality of candidate gene sequences may include summing numerical scores assigned to each of the plurality of allele calls of the second genotyping call data. For example, the numerical values (e.g., “1”) for each positive match between an allele call of the first genotyping call data and a corresponding allele call of the second genotyping call data may be added to determine a match score.

In step 1640, a genotyping call is made for the query gene sequence based on a highest match score from among the match score determined for each of the plurality of candidate gene sequences. For example, as shown in FIG. 15, candidate gene sequence 1530 (sequence A101_003) has a highest match score of “13”, or 100% of the alleles of interest, with respect to input query gene sequence 1310 shown in FIG. 13.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computers and interact via a network. The client-server relationship may be defined and controlled by computer programs running on the respective client and server computers. Examples of client computers can include desktop computers, workstations, portable computers, cellular smartphones, tablets, or other types of computing devices.

Systems, apparatus, and methods described herein may be implemented using a computer program product tangibly embodied in an information carrier, e.g., in a non-transitory machine-readable storage device, for execution by a programmable processor; and the method processes and steps described herein, including one or more of the steps described above with respect to FIGS. 1-19, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an exemplary apparatus that may be used to implement systems, apparatus and methods described herein is illustrated in FIG. 20. Apparatus 2000 comprises a processor 2010 operatively coupled to a persistent storage device 2020 and a main memory device 2030. Processor 2010 controls the overall operation of apparatus 2000 by executing computer program instructions that define such operations. The computer program instructions may be stored in persistent storage device 2020, or other computer-readable medium, and loaded into main memory device 2030 when execution of the computer program instructions is desired. For example, processor 2010 may comprise one or more components of PCR apparatus 100, capillary electrophoresis apparatus 110, and genotyping data analyzer 120. Thus, the method steps described above with respect to FIGS. 1-19 can be defined by the computer program instructions stored in main memory device 2030 and/or persistent storage device 2020 and controlled by processor 2010 executing the computer program instructions. For example, the computer program instructions can be implemented as computer executable code programmed by one skilled in the art to perform an algorithm defined by the method steps described above with respect to FIGS. 1-19. Accordingly, by executing the computer program instructions, the processor 2010 executes an algorithm defined by the method steps described above with respect to FIGS. 1-19. Apparatus 2000 also includes one or more network interfaces 2080 for communicating with other devices via a network. Apparatus 2000 may also include one or more input/output devices 2090 that enable user interaction with apparatus 2000 (e.g., display, keyboard, mouse, speakers, buttons, etc.).

Processor 2010 may include both general and special purpose microprocessors and may be the sole processor or one of multiple processors of apparatus 2000. Processor 2010 may comprise one or more central processing units (CPUs), and one or more graphics processing units (GPUs), which, for example, may work separately from and/or multi-task with one or more CPUs to accelerate processing, e.g., for various image processing applications described herein. Processor 2010, persistent storage device 2020, and/or main memory device 2030 may include, be supplemented by, or incorporated in, one or more application-specific integrated circuits (ASICs) and/or one or more field programmable gate arrays (FPGAs).

Persistent storage device 2020 and main memory device 2030 each comprise a tangible non-transitory computer readable storage medium. Persistent storage device 2020, and main memory device 2030, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 2090 may include peripherals, such as a printer, scanner, display screen, etc. For example, input/output devices 2090 may include a display device such as a cathode ray tube (CRT), plasma or liquid crystal display (LCD) monitor for displaying information to a user, a keyboard, and a pointing device such as a mouse or a trackball by which the user can provide input to apparatus 2000.

Any or all of the functions of the systems and apparatuses discussed herein may be performed by processor 2010, and/or incorporated in, an apparatus such as PCR apparatus 100, capillary electrophoresis apparatus 110, and genotyping data analyzer 120.

One skilled in the art will recognize that an implementation of an actual computer or computer system may have other structures and may contain other components as well, and that FIG. 20 is a high-level representation of some of the components of such a computer for illustrative purposes.

Disclosed is a method of genotyping a gene sequence comprising: obtaining first genotyping call data representing a query gene sequence; assigning a numerical score to each of a plurality of allele calls of second genotyping call data by matching the first genotyping call data with the second genotyping call data, the second genotyping call data representing a plurality of candidate gene sequences; determining a match score for each of the plurality of candidate gene sequences based on the numerical score assigned to each of the plurality of allele calls of the second genotyping call data; and making a genotyping call for the query gene sequence based on a highest match score from among the match scores determined for each of the plurality of candidate gene sequences.

In an embodiment of the method, the obtaining the first genotyping call data comprises obtaining Sanger-based DNA sequencing data representing the query gene sequence; aligning the Sanger-based DNA sequencing data representing the query gene sequence with a reference gene sequence; making an additional genotyping call for each of a plurality of alleles of the query gene sequence based on the aligned Sanger-based DNA sequencing data; and translating the additional genotyping call for each of the plurality of alleles into a code representing the query gene sequence.

In an embodiment of the method, the making of the additional genotyping call comprises generating an electropherogram report of base calls for each of the plurality of alleles of the query gene sequence based on the aligned Sanger-based DNA sequencing data using at least one base caller algorithm; and verifying the base calls for each of the plurality of alleles of the query gene sequence based on an analysis of the electropherogram report.

In an embodiment of the method, the code comprises an International Union of Pure and Applied Chemistry (IUPAC) code, and a heterozygous or homozygous deletion code.

In an embodiment of the method, a first numerical value of “1” is assigned if there is a positive match between an allele call of the first genotyping call data and a corresponding allele call of the second genotyping call data, and a second numerical value of “0” is assigned if there is a non-positive match between the allele call of the first genotyping call data and the corresponding allele call of the second genotyping call data.

In an embodiment of the method, the method further includes generating a look-up table comprising the second genotyping call data, wherein the look-up table comprises a list of codes representing each of the plurality of candidate gene sequences, and wherein the list of codes comprises International Union of Pure and Applied Chemistry (IUPAC) codes, and a heterozygous or homozygous deletion code.

In an embodiment of the method, the matching of the first genotyping call data with the second genotyping call data comprises using at least one find operation to query the look-up table using the first genotyping call data.

In an embodiment of the method, the matching of the first genotyping call data with the second genotyping call data comprises: aligning each allele position of the first genotyping call data with corresponding allele positions of the second genotyping call data; and comparing, at each of the corresponding allele positions of the second genotyping call data, allele calls of the first genotyping call data with the plurality of allele calls of the second genotyping call data.

In an embodiment of the method, the determining of the match score for each of the plurality of candidate gene sequences comprises summing numerical scores assigned to each of the plurality of allele calls of the second genotyping call data.

In an embodiment of the method, the query gene sequence corresponds to a set of variant alleles.

In an embodiment of the method, the first genotyping call data comprises a subset of genotyping call data representing the query gene sequence, and wherein the subset of the genotyping call data corresponds to ABO alleles used for determining major ABO blood types.

In an embodiment of the method, the second genotyping call data is generated based on Sanger-based DNA sequencing data representing the plurality of candidate gene sequences.

In an embodiment of the method, the plurality of candidate gene sequences corresponds to one or more known phenotypes, and the genotyping call for the query gene sequence corresponds to the one or more known phenotypes.

In an embodiment of the method, the one or more known phenotypes comprise at least one ABO phenotype.

Disclosed is a non-transitory computer readable medium comprising a memory storing one or more instructions which, when executed by one or more processors of at least one computing device, perform one or more steps for genotyping a gene sequence by: obtaining first genotyping call data representing a query gene sequence; assigning a numerical score to each of a plurality of allele calls of second genotyping call data by matching the first genotyping call data with the second genotyping call data, the second genotyping call data representing a plurality of candidate gene sequences; determining a match score for each of the plurality of candidate gene sequences based on the numerical score assigned to each of the plurality of allele calls of the second genotyping call data; and making a genotyping call for the query gene sequence based on a highest match score from among the match scores determined for each of the plurality of candidate gene sequences.

In an embodiment in the non-transitory computer readable medium, the obtaining the first genotyping call data comprises: obtaining Sanger-based DNA sequencing data representing the query gene sequence; aligning the Sanger-based DNA sequencing data representing the query gene sequence with a reference gene sequence; making an additional genotyping call for each of a plurality of alleles of the query gene sequence based on the aligned Sanger-based DNA sequencing data; and translating the additional genotyping call for each of the plurality of alleles into a code representing the query gene sequence.

In an embodiment of the non-transitory computer readable medium, the making of the additional genotyping call comprises: generating an electropherogram report of base calls for each of the plurality of alleles of the query gene sequence based on the aligned Sanger-based DNA sequencing data using at least one base caller algorithm; and verifying the base calls for each of the plurality of alleles of the query gene sequence based on an analysis of the electropherogram report.

In an embodiment of the non-transitory computer readable medium, the code comprises an International Union of Pure and Applied Chemistry (IUPAC) code, and a heterozygous or homozygous deletion code.

In an embodiment of the non-transitory computer readable medium, a first numerical value of “1” is assigned if there is a positive match between an allele call of the first genotyping call data and a corresponding allele call of the second genotyping call data, and a second numerical value of “0” is assigned if there is a non-positive match between the allele call of the first genotyping call data and the corresponding allele call of the second genotyping call data.

In an embodiment of the non-transitory computer readable medium, the method further comprises the memory storing one or more instructions which, when executed by one or more processors of at least one computing device, perform one or more steps for genotyping a gene sequence by: generating a look-up table comprising the second genotyping call data, wherein the look-up table comprises a list of codes representing each of the plurality of candidate gene sequences, and wherein the list of codes comprises International Union of Pure and Applied Chemistry (IUPAC) codes, and a heterozygous or homozygous deletion code.

In an embodiment of the non-transitory computer readable medium, the matching of the first genotyping call data with the second genotyping call data comprises using at least one find operation to query the look-up table using the first genotyping call data.

In an embodiment of the non-transitory computer readable medium, the matching of the first genotyping call data with the second genotyping call data comprises: aligning each allele position of the first genotyping call data with corresponding allele positions of the second genotyping call data; and comparing, at each of the corresponding allele positions of the second genotyping call data, allele calls of the first genotyping call data with the plurality of allele calls of the second genotyping call data.

In an embodiment of the non-transitory computer readable medium, the determining of the match score for each of the plurality of candidate gene sequences comprises summing numerical scores assigned to each of the plurality of allele calls of the second genotyping call data.

In an embodiment of the non-transitory computer readable medium, the query gene sequence corresponds to a set of variant alleles.

In an embodiment of the non-transitory computer readable medium, the first genotyping call data comprises a subset of genotyping call data representing the query gene sequence, and wherein the subset of the genotyping call data corresponds to ABO alleles used for determining major ABO blood types.

In an embodiment of the non-transitory computer readable medium, the second genotyping call data is generated based on Sanger-based DNA sequencing data representing the plurality of candidate gene sequences.

In an embodiment of the non-transitory computer readable medium, the plurality of candidate gene sequences corresponds to one or more known phenotypes, and the genotyping call for the query gene sequence corresponds to the one or more known phenotypes.

In an embodiment of the non-transitory computer readable medium, the one or more known phenotypes comprise at least one ABO phenotype.

Disclosed is an apparatus configured for genotyping a gene sequence, the apparatus comprising: one or more processors of at least one computing device; and a memory storing one or more instructions, which, when executed by the one or more processors, cause the one or more processors to perform functions including: obtaining first genotyping call data representing a query gene sequence; assigning a numerical score to each of a plurality of allele calls of second genotyping call data by matching the first genotyping call data with the second genotyping call data, the second genotyping call data representing a plurality of candidate gene sequences; determining a match score for each of the plurality of candidate gene sequences based on the numerical score assigned to each of the plurality of allele calls of the second genotyping call data; and making a genotyping call for the query gene sequence based on a highest match score from among the match scores determined for each of the plurality of candidate gene sequences.

In an embodiment of the apparatus, the obtaining the first genotyping call data comprises: obtaining Sanger-based DNA sequencing data representing the query gene sequence; aligning the Sanger-based DNA sequencing data representing the query gene sequence with a reference gene sequence; making an additional genotyping call for each of a plurality of alleles of the query gene sequence based on the aligned Sanger-based DNA sequencing data; and translating the additional genotyping call for each of the plurality of alleles into a code representing the query gene sequence.

In an embodiment of the apparatus, the making of the additional genotyping call comprises: generating an electropherogram report of base calls for each of the plurality of alleles of the query gene sequence based on the aligned Sanger-based DNA sequencing data using at least one base caller algorithm; and verifying the base calls for each of the plurality of alleles of the query gene sequence based on an analysis of the electropherogram report.

In an embodiment of the apparatus, the code comprises an International Union of Pure and Applied Chemistry (IUPAC) code, and a heterozygous or homozygous deletion code.

In an embodiment of the apparatus, a first numerical value of “1” is assigned if there is a positive match between an allele call of the first genotyping call data and a corresponding allele call of the second genotyping call data, and a second numerical value of “0” is assigned if there is a non-positive match between the allele call of the first genotyping call data and the corresponding allele call of the second genotyping call data.

In an embodiment of the apparatus, the apparatus further comprises causing the one or more processors to perform functions including: generating a look-up table comprising the second genotyping call data, wherein the look-up table comprises a list of codes representing each of the plurality of candidate gene sequences, and wherein the list of codes comprises International Union of Pure and Applied Chemistry (IUPAC) codes, and a heterozygous or homozygous deletion code.

In an embodiment of the apparatus, the matching of the first genotyping call data with the second genotyping call data comprises using at least one find operation to query the look-up table using the first genotyping call data.

In an embodiment of the apparatus, the matching of the first genotyping call data with the second genotyping call data comprises: aligning each allele position of the first genotyping call data with corresponding allele positions of the second genotyping call data; and comparing, at each of the corresponding allele positions of the second genotyping call data, allele calls of the first genotyping call data with the plurality of allele calls of the second genotyping call data.

In an embodiment of the apparatus, the determining of the match score for each of the plurality of candidate gene sequences comprises summing numerical scores assigned to each of the plurality of allele calls of the second genotyping call data.

In an embodiment of the apparatus, the query gene sequence corresponds to a set of variant alleles.

In an embodiment of the apparatus, the first genotyping call data comprises a subset of genotyping call data representing the query gene sequence, and wherein the subset of the genotyping call data corresponds to ABO alleles used for determining major ABO blood types.

In an embodiment of the apparatus, the second genotyping call data is generated based on Sanger-based DNA sequencing data representing the plurality of candidate gene sequences.

In an embodiment of the apparatus, the plurality of candidate gene sequences corresponds to one or more known phenotypes, and the genotyping call for the query gene sequence corresponds to the one or more known phenotypes. In an embodiment of the apparatus, the one or more known phenotypes comprise at least one ABO phenotype.

Disclosed is a method of genotyping a gene sequence, the method comprising: providing a sample comprising the gene sequence; amplifying the gene sequence using a primer pair of SEQ ID NOs. 7-8 or any derivative sequence of SEQ ID NOs. 7-8; determining one or more base sequences of the gene sequence; and making a genotyping call based on the determined base sequences.

In an embodiment of the method, the method further comprises amplifying the gene sequence using one or more primer pairs selected from the group consisting of SEQ ID NOs. 1-6 or any derivative sequences thereof.

In an embodiment of the method, the derivate sequence comprises a sequence identity of about or at least 70%, about or at least 75%, about or at least 80%, about or at least 85%, about or at least 90%, about or at least 95%, about or at least 96%, about or at least 97%, about or at least 98%, or about or at least 99% to sequences of SEQ ID NOs. 1-8.

In an embodiment of the method, the one or more base sequences are determined via Sanger sequencing.

Disclosed is a composition comprising one or more sequences selected from a group consisting of SEQ ID NOs. 7-8 and any derivative thereof.

In an embodiment of the composition, the composition further comprises one or more sequence selected from the group consisting of SEQ ID NOs. 1-6 or any derivative sequences thereof.

In an embodiment of the composition, the derivate sequence comprises a sequence identity of about or at least 70%, about or at least 75%, about or at least 80%, about or at least 85%, about or at least 90%, about or at least 95%, about or at least 96%, about or at least 97%, about or at least 98%, or about or at least 99% to sequences of SEQ ID NOs. 1-8.

Disclosed is a kit for genotyping, the kit comprising: one or more sequences selected from a group consisting of SEQ ID NOs. 7-8 and any derivative thereof; DNA polymerase; and a buffer.

In an embodiment of the kit, the kit further comprises one or more sequences selected from a group consisting of SEQ ID NOs. 1-6 or any derivative sequences thereof.

In an embodiment of the kit, the derivate sequence comprises a sequence identity of about or at least 70%, about or at least 75%, about or at least 80%, about or at least 85%, about or at least 90%, about or at least 95%, about or at least 96%, about or at least 97%, about or at least 98%, or about or at least 99% to sequences of SEQ ID NOs. 1-8.

The foregoing specification is to be understood as being in every respect illustrative and exemplary, but not restrictive, and the scope of the invention disclosed herein is not to be determined from the specification, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

### INDUSTRIAL APPLICABILITY

The various embodiments herein find industrial application in DNA sequencing at specific positions within the genome of an individual and improving the accuracy of PCR, Sanger sequencing, and capillary electrophoresis methods and systems in applications such as genotyping and rare allele detection. In addition, the primer pairs selected from the group consisting of SEQ ID NOs. 1-8, or any derivative sequences thereof, have industrial applicability which can include their use in, inter alia, the amplification by PCR of nucleotide sequences, and the identification and characterization of a gene or genes of interest.

### SEQUENCE LISTING

The contents of the electronic sequence listing (101212007740TP109239WO1PCTsequencelisting.xml; Size: 8,258 bytes; and Date of Creation: Oct. 17, 2022) submitted herewith is herein incorporated by reference in its entirety.

What is claimed is:

1. A method of genotyping a gene sequence, the method comprising:
obtaining first genotyping call data representing a query gene sequence;
assigning a numerical score to each of a plurality of allele calls of second genotyping call data by matching the first genotyping call data with the second genotyping call data, the second genotyping call data representing a plurality of candidate gene sequences;
determining a match score for each of the plurality of candidate gene sequences based on the numerical score assigned to each of the plurality of allele calls of the second genotyping call data; and
making a genotyping call for the query gene sequence based on a highest match score from among the match scores determined for each of the plurality of candidate gene sequences.

2. The method of claim 1, wherein the obtaining the first genotyping call data comprises:
obtaining Sanger-based DNA sequencing data representing the query gene sequence;
aligning the Sanger-based DNA sequencing data representing the query gene sequence with a reference gene sequence;
making an additional genotyping call for each of a plurality of alleles of the query gene sequence based on the aligned Sanger-based DNA sequencing data; and
translating the additional genotyping call for each of the plurality of alleles into a code representing the query gene sequence.

3. The method of claim 2, wherein the making of the additional genotyping call comprises:
generating an electropherogram report of base calls for each of the plurality of alleles of the query gene sequence based on the aligned Sanger-based DNA sequencing data using at least one base caller algorithm; and
verifying the base calls for each of the plurality of alleles of the query gene sequence based on an analysis of the electropherogram report.

4. The method of any of claims 2-3, wherein the code comprises an International Union of Pure and Applied Chemistry (IUPAC) code, and a heterozygous or homozygous deletion code.

5. The method of any of claims 1-4, wherein a first numerical value of “1” is assigned if there is a positive match between an allele call of the first genotyping call data and a corresponding allele call of the second genotyping call data, and a second numerical value of “0” is assigned if there is a non-positive match between the allele call of the first genotyping call data and the corresponding allele call of the second genotyping call data.

6. The method of any of claims 1-5, further comprising:
generating a look-up table comprising the second genotyping call data, wherein the look-up table comprises a list of codes representing each of the plurality of candidate gene sequences, and wherein the list of codes comprises International Union of Pure and Applied Chemistry (IUPAC) codes, and a heterozygous or homozygous deletion code.

7. The method of claim 6, wherein the matching of the first genotyping call data with the second genotyping call data comprises using at least one find operation to query the look-up table using the first genotyping call data.

8. The method of claim 1, wherein the matching of the first genotyping call data with the second genotyping call data comprises:
aligning each allele position of the first genotyping call data with corresponding allele positions of the second genotyping call data; and
comparing, at each of the corresponding allele positions of the second genotyping call data, allele calls of the first genotyping call data with the plurality of allele calls of the second genotyping call data.

9. The method of any of claims 1-8, wherein the determining of the match score for each of the plurality of candidate gene sequences comprises summing numerical scores assigned to each of the plurality of allele calls of the second genotyping call data.

10. The method of any of claims 1-9, wherein the query gene sequence corresponds to a set of variant alleles.

11. The method of any of claims 1-10, wherein the first genotyping call data comprises a subset of genotyping call data representing the query gene sequence, and wherein the subset of the genotyping call data corresponds to ABO alleles used for determining major ABO blood types.

12. The method of any of claims 1-11, wherein the second genotyping call data is generated based on Sanger-based DNA sequencing data representing the plurality of candidate gene sequences.

13. The method of any of claims 1-12, wherein the plurality of candidate gene sequences corresponds to one or more known phenotypes, and wherein the genotyping call for the query gene sequence corresponds to the one or more known phenotypes.

14. The method of claim 13, wherein the one or more known phenotypes comprise at least one ABO phenotype.

15. A non-transitory computer readable medium comprising a memory storing one or more instructions which, when executed by one or more processors of at least one computing device, perform one or more steps for genotyping a gene sequence by:
obtaining first genotyping call data representing a query gene sequence;
assigning a numerical score to each of a plurality of allele calls of second genotyping call data by matching the first genotyping call data with the second genotyping call data, the second genotyping call data representing a plurality of candidate gene sequences;
determining a match score for each of the plurality of candidate gene sequences based on the numerical score assigned to each of the plurality of allele calls of the second genotyping call data; and
making a genotyping call for the query gene sequence based on a highest match score from among the match scores determined for each of the plurality of candidate gene sequences.

16. The computer readable medium of claim 15, wherein the obtaining the first genotyping call data comprises:
obtaining Sanger-based DNA sequencing data representing the query gene sequence;
aligning the Sanger-based DNA sequencing data representing the query gene sequence with a reference gene sequence;
making an additional genotyping call for each of a plurality of alleles of the query gene sequence based on the aligned Sanger-based DNA sequencing data; and
translating the additional genotyping call for each of the plurality of alleles into a code representing the query gene sequence.

17. The computer readable medium of claim 16, wherein the making of the additional genotyping call comprises:
generating an electropherogram report of base calls for each of the plurality of alleles of the query gene sequence based on the aligned Sanger-based DNA sequencing data using at least one base caller algorithm; and
verifying the base calls for each of the plurality of alleles of the query gene sequence based on an analysis of the electropherogram report.

18. The computer readable medium of any of claims 16-17, wherein the code comprises an International Union of Pure and Applied Chemistry (IUPAC) code, and a heterozygous or homozygous deletion code.

19. The computer readable medium of any of claims 15-18, wherein a first numerical value of “1” is assigned if there is a positive match between an allele call of the first genotyping call data and a corresponding allele call of the second genotyping call data, and a second numerical value of “0” is assigned if there is a non-positive match between the allele call of the first genotyping call data and the corresponding allele call of the second genotyping call data.

20. The computer readable medium of any of claims 15-19, further comprising the memory storing one or more instructions which, when executed by one or more processors of at least one computing device, perform one or more steps for genotyping a gene sequence by:
generating a look-up table comprising the second genotyping call data,
wherein the look-up table comprises a list of codes representing each of the plurality of candidate gene sequences, and
wherein the list of codes comprises International Union of Pure and Applied Chemistry (IUPAC) codes, and a heterozygous or homozygous deletion code.

21. The computer readable medium of claim 20, wherein the matching of the first genotyping call data with the second genotyping call data comprises using at least one find operation to query the look-up table using the first genotyping call data.

22. The computer readable medium of claim 15, wherein the matching of the first genotyping call data with the second genotyping call data comprises:
aligning each allele position of the first genotyping call data with corresponding allele positions of the second genotyping call data; and
comparing, at each of the corresponding allele positions of the second genotyping call data, allele calls of the first genotyping call data with the plurality of allele calls of the second genotyping call data.

23. The computer readable medium of any of claims 15-22, wherein the determining of the match score for each of the plurality of candidate gene sequences comprises summing numerical scores assigned to each of the plurality of allele calls of the second genotyping call data.

24. The computer readable medium of any of claims 15-23, wherein the query gene sequence corresponds to a set of variant alleles.

25. The computer readable medium of any of claims 15-24, wherein the first genotyping call data comprises a subset of genotyping call data representing the query gene sequence, and wherein the subset of the genotyping call data corresponds to ABO alleles used for determining major ABO blood types.

26. The computer readable medium of any of claims 15-25, wherein the second genotyping call data is generated based on Sanger-based DNA sequencing data representing the plurality of candidate gene sequences.

27. The computer readable medium of any of claims 15-26, wherein the plurality of candidate gene sequences corresponds to one or more known phenotypes, and wherein the genotyping call for the query gene sequence corresponds to the one or more known phenotypes.

28. The computer readable medium of claim 27, wherein the one or more known phenotypes comprise at least one ABO phenotype.

29. An apparatus configured for genotyping a gene sequence, the apparatus comprising:
one or more processors of at least one computing device; and
a memory storing one or more instructions, which, when executed by the one or more processors, cause the one or more processors to perform functions including:
obtaining first genotyping call data representing a query gene sequence;
assigning a numerical score to each of a plurality of allele calls of second genotyping call data by matching the first genotyping call data with the second genotyping call data, the second genotyping call data representing a plurality of candidate gene sequences;
determining a match score for each of the plurality of candidate gene sequences based on the numerical score assigned to each of the plurality of allele calls of the second genotyping call data; and
making a genotyping call for the query gene sequence based on a highest match score from among the match scores determined for each of the plurality of candidate gene sequences.

30. The apparatus of claim 29, wherein the obtaining the first genotyping call data comprises:
obtaining Sanger-based DNA sequencing data representing the query gene sequence;
aligning the Sanger-based DNA sequencing data representing the query gene sequence with a reference gene sequence;
making an additional genotyping call for each of a plurality of alleles of the query gene sequence based on the aligned Sanger-based DNA sequencing data; and
translating the additional genotyping call for each of the plurality of alleles into a code representing the query gene sequence.

31. The apparatus of claim 30, wherein the making of the additional genotyping call comprises:
generating an electropherogram report of base calls for each of the plurality of alleles of the query gene sequence based on the aligned Sanger-based DNA sequencing data using at least one base caller algorithm; and
verifying the base calls for each of the plurality of alleles of the query gene sequence based on an analysis of the electropherogram report.

32. The apparatus of any of claims 30-31, wherein the code comprises an International Union of Pure and Applied Chemistry (IUPAC) code, and a heterozygous or homozygous deletion code.

33. The apparatus of any of claims 29-32, wherein a first numerical value of “1” is assigned if there is a positive match between an allele call of the first genotyping call data and a corresponding allele call of the second genotyping call data, and a second numerical value of “0” is assigned if there is a non-positive match between the allele call of the first genotyping call data and the corresponding allele call of the second genotyping call data.

34. The apparatus of any of claims 29-33, further comprising causing the one or more processors to perform functions including:
generating a look-up table comprising the second genotyping call data,
wherein the look-up table comprises a list of codes representing each of the plurality of candidate gene sequences, and
wherein the list of codes comprises International Union of Pure and Applied Chemistry (IUPAC) codes, and a heterozygous or homozygous deletion code.

35. The apparatus of claim 34, wherein the matching of the first genotyping call data with the second genotyping call data comprises using at least one find operation to query the look-up table using the first genotyping call data.

36. The apparatus of claim 29, wherein the matching of the first genotyping call data with the second genotyping call data comprises:
aligning each allele position of the first genotyping call data with corresponding allele positions of the second genotyping call data; and
comparing, at each of the corresponding allele positions of the second genotyping call data, allele calls of the first genotyping call data with the plurality of allele calls of the second genotyping call data.

37. The apparatus of any of claims 29-36, wherein the determining of the match score for each of the plurality of candidate gene sequences comprises summing numerical scores assigned to each of the plurality of allele calls of the second genotyping call data.

38. The apparatus of any of claims 29-37, wherein the query gene sequence corresponds to a set of variant alleles.

39. The apparatus of any of claims 29-38, wherein the first genotyping call data comprises a subset of genotyping call data representing the query gene sequence, and wherein the subset of the genotyping call data corresponds to ABO alleles used for determining major ABO blood types.

40. The apparatus of any of claims 29-39, wherein the second genotyping call data is generated based on Sanger-based DNA sequencing data representing the plurality of candidate gene sequences.

41. The apparatus of any of claims 29-40, wherein the plurality of candidate gene sequences corresponds to one or more known phenotypes, and
wherein the genotyping call for the query gene sequence corresponds to the one or more known phenotypes.

42. The apparatus of claim 41, wherein the one or more known phenotypes comprise at least one ABO phenotype.

43. A method of genotyping a gene sequence, the method comprising:
providing a sample comprising the gene sequence;
amplifying the gene sequence using a primer pair of SEQ ID NOs. 7-8 or any derivative sequence of SEQ ID NOs. 7-8;
determining one or more base sequences of the gene sequence; and
making a genotyping call based on the determined base sequences.

44. The method of claim 43, wherein the method further comprises amplifying the gene sequence using one or more primer pairs selected from the group consisting of SEQ ID NOs. 1-6 or any derivative sequences thereof.

45. The method of any of claims 43-44, wherein the derivate sequence comprises a sequence identity of about or at least 70%, about or at least 75%, about or at least 80%, about or at least 85%, about or at least 90%, about or at least 95%, about or at least 96%, about or at least 97%, about or at least 98%, or about or at least 99% to sequences of SEQ ID NOs. 1-8.

46. The method of claim 43, wherein the one or more base sequences are determined via Sanger sequencing.

47. A composition comprising one or more sequences selected from a group consisting of SEQ ID NOs. 7-8 and any derivative thereof.

48. The composition of claim 47, wherein the composition further comprises one or more sequence selected from the group consisting of SEQ ID NOs. 1-6 or any derivative sequences thereof.

49. The composition of any of claims 47-48, wherein the derivate sequence comprises a sequence identity of about or at least 70%, about or at least 75%, about or at least 80%, about or at least 85%, about or at least 90%, about or at least 95%, about or at least 96%, about or at least 97%, about or at least 98%, or about or at least 99% to sequences of SEQ ID NOs. 1-8.

50. A kit for genotyping, the kit comprising:
one or more sequences selected from a group consisting of SEQ ID NOs. 7-8 and any derivative thereof;
DNA polymerase; and
a buffer.

51. The kit of claim 50, wherein the kit further comprises one or more sequences selected from a group consisting of SEQ ID NOs. 1-6 or any derivative sequences thereof.

52. The kit of any of claims 50-51, wherein the derivate sequence comprises a sequence identity of about or at least 70%, about or at least 75%, about or at least 80%, about or at least 85%, about or at least 90%, about or at least 95%, about or at least 96%, about or at least 97%, about or at least 98%, or about or at least 99% to sequences of SEQ ID NOs. 1-8.
